(19) United States
(12) Patent Application Publication
(10) Pub. No.: US 2020/0115419 A1
(43) Pub. Date: Apr. 16, 2020

(54) NEW OPTOGENETIC TOOL

(71) Applicant: MAX-PLANCK-GESELLSCHAFT ZUR FÖRDERUNG DER WISSENSCHAFTEN E.V., München (DE)

(72) Inventors: Ernst Bamberg, Kelkheim (DE); Valentin Gordeliy, Laffrey (FR); Thomas Mager, Frankfurt (DE); Vitaly Shevchenko, Jülich (DE)

(73) Assignee: MAX-PLANCK-GESELLSCHAFT ZUR FÖRDERUNG DER WISSENSCHAFTEN E.V., München (DE)

(21) Appl. No.: 16/603,963

(22) PCT Filed: Apr. 11, 2018
(86) PCT No.: PCT/EP2018/059297

§ 371 Date: Oct. 9, 2019

(30) Foreign Application Priority Data
Apr. 12, 2017 (EP) 17166302.4

(51) Int. Cl. C07K 14/00; A61K 9/127; A61N 5/06

(52) U.S. Cl. CPC C07K 14/00; A61K 9/127; A61K 38/00; A61N 5/0622; A61N 5/062

(57) ABSTRACT

The invention relates to newly characterized light-inducible inward proton pumps and their use in medicine, their utility as optogenetic tools, nucleic acid constructs encoding same, expression vectors carrying the nucleic acid construct, cells comprising said nucleic acid construct or expression vector, and their respective uses.

The invention relates to newly characterized light-inducible inward proton pumps and their use in medicine, their utility as optogenetic tools, nucleic acid constructs encoding same, expression vectors carrying the nucleic acid construct, cells comprising said nucleic acid construct or expression vector, and their respective uses.

### BACKGROUND OF THE INVENTION

All cells maintain a particular concentration of ions H⁺, K⁺, Na⁺ and Cl⁻ in cytoplasm, which is crucial for life. Ion gradients across cell membranes are maintained by ion transporters which are integral membrane proteins. In many archaea, bacteria and unicellular eukaryotes, these gradients are created (among other mechanisms) by light-driven microbial rhodopsins, seven transmembrane α-helix proteins comprising a co-factor chromophore named retinal. Proton gradients are maintained by outward proton pumps and they play a crucial role in providing energy for most of biochemical reactions. Although a light-driven proton pumping rhodopsin (bacteriorhodopsin) was discovered long ago in archaea (Oesterhelt, D. & Stoeckenius, W. Rhodopsin-like Protein from the Purple Membrane of Halobacterium halobium. Nature 233, 149-152 (1971)), and later in other domains of life and they have the widest ecological distribution in soil, hypersaline, marine and freshwater habitats, all known proton pumping rhodopsins are outward directed (Ernst, O. P. et al. Microbial and Animal Rhodopsins: Structures, Functions, and Molecular Mechanisms. Chem. Rev. 114, 126-163 (2014)). The same is true for non-rhodopsin proton pumps. Inwardly directed cell proton pumps have not been known thus far. Their existence has not been even discussed. Despite the fact that inward cell organelle proton pumps like Na⁺/H⁺ anti-porter are known, inward plasma membrane proton pumps are yet completely unknown.

In 2011, a new class of microbial rhodopsins, distinct from other rhodopsin types, was discovered (Ugalde, J. A., Podell, S., Narasingarao, P. & Allen, E. E. Xenorhodopsins, an enigmatic new class of microbial rhodopsins horizontally transferred between archaea and bacteria. Biol. Direct 6, 52 (2011)). The authors of that work found several new homologues of Anabaena sensory rhodopsin (ASR)5. The members of the class were named xenorhodopsins (XeRs). Among these proteins were xenorhodopsins from a new major lineage of Archaea, specifically the nanohaloarchaeon Nanosalina sp. j07AB43 and Nanosalinarum sp. J07AB56 with 89% of amino acid identity between them but with only 34% identity to ASR. Analyzing the amino acid sequence alignment the authors concluded that xenorhodopsins do not share specific features common to proton- or halorhodopsin pumps. However, they are similar to ASR since they lack a common Asp at the donor position like the sensory rhodopsins known at that time. Therefore, Ugalde et al. speculate that xenorhodopsins have a function similar to sensory rhodopsins. At the same time, Ugalde et al. admit that no sensory or ion transport function has yet been experimentally validated for ASR, or any other xenorhodopsin protein.

Kawanabe et al. reported the artificial ASR mutant D217E, which exhibited a light-driven inward proton transport activity (Kawanabe et al. Engineering an inward proton transport from a bacterial sensor rhodopsin. J Am Chem Soc. 131, 16439-16444 (2009); Kawanabe et al. An inward proton transport using Anabaena sensory rhodopsin. J Microbiol. 49, 1-6 (2011)). See also Dong et al. Structure of an Inward Proton-Transporting Anabaena Sensory Rhodopsin Mutant: Mechanistic Insights. Biophys J. 111, 963-972 (September 2016). However, Kawanabe et al. showed that the efficiency of inward proton transport by D217E ASR is low (15 times lower than the efficiency of BR). Moreover Kawanabe et al. did not show if D217E ASR functioned as a H⁺ pump or channel. In contrast the xenorhodopsins described and characterized herein are proven to be light-driven inward proton pumps which allow highly efficient proton transport.

Inoue et al. describe the production of a blue-shifted, light-gated proton channel (AR3-T) by replacing three residues located around the retinal (i.e. M128A, G132V, and A225T) in the light-driven outward proton pump archaerhodopsin-3 (AR-3) (Inoue et al. Converting a light-driven proton pump into a light-gated proton channel. J Am Chem Soc. 137, 3291-3299 (2015)). The light-gated proton channel AR3-T does not allow proton transport against an electrochemical gradient. It is also reported that AR3-T has a very slow photocycle, which makes AR3-T unsuitable for several optogenetic applications.

Recently, Inoue et al. also reported the discovery of an inward H⁺ pump from Parvularcula oceani (Inoue et al. A natural light-driven inward proton pump. Nat Commun. 7, 13415 (November 2016), and the expression of same in Escherichia coli and in mouse neural cells. However, the light induced depolarizing current by the inward H⁺ pump from Parvularcula oceani is insufficient for activation of neuronal cells. Of note the reported kinetics are considerably slower as compared to the xenorhodopsins described and characterized herein, which generally limits the use of the inward H⁺ pump from Parvularcula oceani as an optogenetic tool and precludes the possibility of neuronal activation with high temporal accuracy.

Three proteins from the xenorhodopsin family, i.e. NsXeR (sequence disclosed in Ugalde, et al. Biol. Direct 6, 52 (2011)), HrvXeR (sequence disclosed in Ghai, R. et al. New Abundant Microbial Groups in Aquatic Hypersaline Environments. Sci. Rep. 1, (2011)) and AlkXeR (sequence disclosed in Vavourakis, C. D. et al. Metagenomic Insights into the Uncultured Diversity and Physiology of Microbes in Four Hypersaline Soda Lake Brines. Front. Microbiol. 7, (2016)), were characterized for the first time, and it was found that all the three are inwardly directed proton pumps. With one of these proteins, a comprehensive function and structure study was performed, the results of which are described below. Moreover, it was found that the light induced depolarizing current by NsXeR is sufficient for reliable activation of neuronal cell with high temporal accuracy. Therefore, NsXeR as a proton pump is attractive for optogenetic studies because the cation independent activity and represents an alternative to the well-known cation selective channelrhodopsins.

It is an object of the present disclosure to provide new optogenetic tools, which are cation independent, pH insensitive, and can be expressed in a broad spectrum of cells. Such optogenetic tools are considered valuable in the field of scientific research as well as in medicine.

### SUMMARY OF THE INVENTION

Generation of electrochemical proton gradient is the first and universal step of cell bioenergetics. In prokaryotes the gradient is created by outward membrane protein proton pumps. Inward plasma membrane native proton pumps were yet unknown. In the present disclosure, comprehensive functional studies of the representatives of the yet non-characterized xenorhodopsins from Nanohaloarchaea family of microbial rhodopsins are described. It is demonstrated in the examples herein for the first time that they are inward proton pumps in model membrane systems, E. coli cells, human embryonic kidney cells, neuroblastoma cells and rat hippocampal neuronal cells. It is demonstrated that the NsXeR is a powerful pump which is able to elicit action potentials in rat hippocampal neuronal cells up to their maximal intrinsic firing frequency, proving that the inwardly directed proton pumps are suitable for light induced remote control of neurons and are an alternative to the well-known cation selective channelrhodopsins.

Accordingly, disclosed is a light-driven inward directed proton pump having at least 59% sequence similarity over the full length of SEQ ID NO: 1 (NsXeR) for use in medicine, as further defined in the claims. For example, the light-driven inward directed proton pump may comprise or consist of an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5).

Also provided is a nucleic acid construct, comprising a nucleotide sequence coding for the light-driven inward directed proton pump as disclosed herein, wherein the nucleotide sequence is codon-optimized for expression in human cells; and an expression vector, comprising a nucleotide sequence coding for light-driven inward directed proton pump as disclosed herein or said nucleic acid construct, wherein the nucleotide sequence is optimized for expression in human cells.

Also contemplated is a mammalian cell expressing the light-driven inward directed proton pump as disclosed herein, with the proviso that the mammalian cell is not a human embryonic cell or a cell capable of modifying the germ line genetic identity of human beings; and a mammalian cell comprising the nucleic acid construct or the expression vector of the present disclosure. Furthermore, the present disclosure also provides a liposome, comprising the light-driven inward directed proton pump as disclosed herein.

The light-driven inward directed proton pump, the nucleic acid construct, the expression vector, the mammalian cell, or the liposome of the present disclosure may be advantageously used in medicine, such as for use in restoring auditory activity, recovery of vision, or for use in treating or alleviating alkalosis, neurological injury, brain damage, seizure, or a degenerative neurological disorder, such as Parkinson's disease and Alzheimer's disease.

In addition, the present disclosure provides a non-human mammal, comprising a cell of the present disclosure, preferably wherein the cell is an endogenous cell; with the proviso that those animals are excluded, which are not likely to yield in substantial medical benefit to man or animal which will outweigh any animal suffering.

Finally, also provided is a non-therapeutic, or ex vivo, or in vitro use of a light-driven inward directed proton pump as disclosed herein, (i) for light-stimulation of electrically excitable cells, (ii) for transporting protons over a membrane against a proton concentration gradient, (iii) for acidifying or alkalinizing the interior of a cell, cell compartment, vesicle, or liposome, or (iv) or as an optogenetic tool.

### DETAILED DESCRIPTION OF THE PREFERRED EMBODIMENTS

The examples herein show comprehensive functional studies of the representatives of the yet non-characterized xenorhodopsins from the Nanohaloarchaea family of microbial rhodopsins and show that they are inwardly directed proton pumps. A rigorous study of the pumping activity of xenorhodopsin from Nanosalina (NsXeR) in model membrane systems, E. coli cells, human embryonic kidney cells, neuroblastoma glioma cells and rat hippocampal neuronal cells confirmed that in all these cells NsXeR works as an inwardly directed pump. It is also demonstrated that the NsXeR is a powerful pump with a turnover rate of 400 s⁻¹ which is able to elicit action potentials in rat hippocampal neuronal cells up to their maximal intrinsic firing frequency. The crystallographic structure of NsXeR reveals the ion translocation pathway that is very different from that of the known rhodopsins. Due to its intrinsic properties as a proton pump NsXeR is completely independent of the ion conditions, which makes this rhodopsin an attractive alternative for light induced remote control of neurons as the well-known cation selective channelrhodopsins.

Accordingly, disclosed herein is a light-driven inward directed proton pump having at least 59% sequence similarity over the full length of SEQ ID NO: 1 (NsXeR) for use in medicine. In preferred embodiments, the light-driven inward directed proton pump has at least 65%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably 99% sequence similarity to the full length of SEQ ID NO: 1 (NsXeR).

Alternatively, or in addition, the light-driven inward directed proton pump can have at least 38%, more preferably at least 45%, more preferably at least 48%, more preferably at least 50%, more preferably at least 55%, more preferably at least 56%, more preferably at least 57%, more preferably at least 58%, more preferably at least 59%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% sequence identity to the full length of SEQ ID NO: 1 (NsXeR).

Generally, an amino acid sequence has “at least x % identity” with another amino acid sequence, e.g. SEQ ID NO: 1 above, when the sequence identity between those to aligned sequences is at least x % over the full length of said other amino acid sequence, e.g. SEQ ID NO: 1. Similarly, an amino acid sequence has “at least x % similarity” with another amino acid sequence, e.g. SEQ ID NO: 1 above, when the sequence similarity between those two aligned sequences is at least x % over the full length of said other amino acid sequence, e.g. SEQ ID NO: 1.

Such alignments can be performed using for example publicly available computer homology programs such as the “EMBOSS” program provided at the EMBL homepage at http://www.ebi.ac.uk/Tools/psa/emboss_needle/, using the default settings provided therein. Further methods of calculating sequence identity or sequence similarity percentages of sets of amino acid acid sequences are known in the art.

The light-driven inward proton pump has seven transmembrane α-helices (A-G) and a co-factor retinal covalently bound to the residue corresponding to 213 Lysine in SEQ ID NO: 1 via the Schiff base. The helix A is preceded with a small N-terminal α-helix, which is capping the protein on the extracellular side.

The light-driven inward directed proton pump of the present disclosure is a membrane protein with at least 5 transmembrane helices, which is capable of binding a light-sensitive polyene. Transmembrane proteins with 6 or 7 transmembrane helices are preferable. Transmembrane proteins with more than 7 helices, for example 8, 9 or 10 transmembrane helices, are however also encompassed. Furthermore, the invention covers transmembrane proteins which in addition to the transmembrane part include C- and/or N-terminal sequences, where the C-terminal sequences can extend into the inside of the lumen enclosed by the membrane, for example the cytoplasm of a cell or the inside of a liposome, or can also be arranged on the membrane outer surface. The same applies for the optionally present N-terminal sequences, which can likewise be arranged both within the lumen and also on the outer surface of the membrane. The length of the C- and/or N-terminal sequences is in principle subject to no restriction; however, light-driven inward directed proton pumps with C-terminal sequences not embedded in the membrane, with 1 to 1000 amino acids, preferably 1 to 500, especially preferably 5 to 50 amino acids, are preferred. Independently of the length of the C-terminal sequences, the N-terminal located sequences not embedded in the membrane preferably comprise 1 to 500 amino acids, especially preferably 5 to 50 amino acids. In a preferred embodiment, the light-driven inward directed proton pump is not truncated at the N-terminus. The concept of the transmembrane helix is well known to the skilled person. These are generally α-helical protein structures, which as a rule comprise 20 to 25 amino acids. However, depending on the nature of the membrane, which can be a natural membrane, for example a cell or plasma membrane, or also a synthetic membrane, the transmembrane segments can also be shorter or longer. For example, transmembrane segments in artificial membranes can comprise up to 30 amino acids, but on the other hand also only a few amino acids, for example 12 to 16.

Most preferably, the light-driven inward proton pump XeR has seven transmembrane α-helices (A-G) and a co-factor retinal covalently bound to 213 Lysine via the Schiff base. The helix A is preceded with a small N-terminal α-helix, which is capping the protein on the extracellular side.

Preferably, the light-driven inward directed proton pump only comprises (semi)-conservative substitutions as compared to SEQ ID NO: 1. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Typical semi-conservative and conservative substitutions are:

| | Conservative | Semi-conservative |
|---|---|---|
| Amino acid | substitution | substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Furthermore, the skilled person will appreciate that glycines at sterically demanding positions should not be substituted and that proline should not be introduced into parts of the protein which have an alpha-helical or a beta-sheet structure. In particular, the light-driven inward directed proton pump may not be mutated at the position corresponding to E4, H48, S55, W73, D76, S80, A87, P209, C212, K214, and D220 of SEQ ID NO: 1. In other words, the light-driven inward directed proton pump preferably comprises an “E” at position 4, an “H” at position 48, etc.

In an even more preferred embodiment, the light-driven inward directed proton pump comprises an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5); in particular wherein the light-driven inward directed proton pump comprises the amino acid sequence of SEQ ID NO: 1 (NsXeR). In a most preferred embodiment, the light-driven inward directed proton pump consists of an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5); in particular wherein the light-driven inward directed proton pump consists of the amino acid sequence of SEQ ID NO: 1 (NsXeR).

The term “inward directed” as used herein is intended to mean that when the proton pump is expressed in a cell, and incorporated into the cell's membrane, it transfers protons (even against a gradient) inwards into the cell. The functional requirement of being “a light-driven inward directed proton pump” can be tested using the following assay. Purified candidate protein is reconstituted in soybean liposomes as described previously (Huang, K. S., Bayley, H. & Khorana, H. G. Delipidation of bacteriorhodopsin and reconstitution with exogenous phospholipid. Proc. Natl. Acad. Sci. 77, 323-327 (1980); incorporated herein by reference). Briefly, phospholipids (asolectin from soybean, Sigma-Aldrich) are dissolved in CHCl₃ (Chloroform ultrapure, Applichem Panreac) and dried under a stream of N₂ in a glass vial. Residual solvent is removed with a vacuum pump overnight. The dried lipids are resuspended at a final concentration of 1% (w/v) in 0.15 M NaCl supplemented with 2% (w/v) sodium cholate. The mixture is clarified by sonication at 4° C. and xenorhodopsin is added at a protein/lipid ratio of 7:100 (w/w). The detergent is removed by overnight stirring with detergent-absorbing beads (Amberlite XAD 2, Supelco). The mixture is dialyzed against 0.15 M NaCl adjusted to pH 7 at 4° C. for 1 day (four 200 ml changes). The measurements are performed on 2 ml of stirred proteoliposome suspension at 0° C. Proteoliposomes are illuminated for 18 minutes with a halogen lamp (Intralux 5000-1, VOLPI) and are then were kept in the dark for another 18 minutes. Changes in pH are monitored with a pH meter (LAB 850, Schott Instruments). As a negative control, measurements are repeated in the presence of 40 uM of CCCP under the same conditions. In case of an inwardly directed proton pumpm the pH changes upon illumination show acidification of the solution outside the membrane. These pH changes are abolished, when CCCP is added to the suspension.

In certain embodiments, wherein the light-driven inward directed proton pump is active between pH 6 and pH 8; preferably between pH 5 and pH 9. This feature may be tested using the foregoing liposome assay, but adjusting the proteoliposomes via dialysis to a starting pH other than pH 7.0.

Using the foregoing liposome assay, one can also illuminate with light of different wavelengths. The light-driven inwardly directed proton pump is typically characterized by exhibiting an the absorption maximum between 560 nm and 580 nm. See also Example 2 below.

However, the light-driven inwardly directed proton pump of the present disclosure can also be further characterized in terms of its photocycle. Preferably, the photocycle of the light-driven inward directed proton pump is less than 50 ms, preferably less than 45 ms, more preferably less than 40 ms, more preferably less than 35 ms, even more preferably less than 30 ms, such as 27 ms, if measured in proteo-nanodiscs exhibiting a molar ratio of DMPC:MSP1E3:light-driven inward directed proton pump of 100:2:3 at 20° C. and pH 7.5, providing pulses of 5 ns duration at 532 nm wavelength and energy of 3 mJ/pulse.

Briefly, the proteo-nanodiscs are assembled using a standard protocol (Ritchie, T. K. et al. in Methods in Enzymology (ed. Düzgünes, N.) 464, 211-231 (Academic Press, 2009); incorporated herein by reference). 1,2-dimyristoyl-sn-glycero-3-phosphocholine, DMPC (Avanti Polar Lipids, USA) is used as lipid. An elongated MSP1E3 version of apolipoprotein-1 is used. The molar ratio during assembly is DMPC:MSP1E3:NsXeR=100:2:3. Liposomes are prepared as described above.

The absorption spectra are recorded using the Shimadzu UV-2401 PC spectrophotometer. The laser flash photolysis setup is similar to that described by Chizhov and co-workers (Chizhov, I. et al. Spectrally silent transitions in the bacteriorhodopsin photocycle. Biophys. J. 71, 2329-2345 (1996); incorporated herein by reference). The excitation/detection systems are composed as such: a Surelite II-10 Nd:YAG laser (Continuum Inc, USA) is used providing pulses of 5 ns duration at 532 nm wavelength and energy of 3 mJ/pulse. Samples (5×5 mm spectroscopic quartz cuvette (Hellma GmbH & Co, Germany)) are placed in a thermostated house between two collimated and mechanically coupled monochromators (⅛ m model 77250, Oriel Corp., USA). The probing light (Xe-arc lamp, 75 W, Osram, Germany) passes the first monochromator, sample and arrives after a second monochromator at a PMT detector (R3896, Hamamatzu, Japan). The current-to-voltage converter of the PMT determines the time resolution of the measurement system of ca 50 ns (measured as an apparent pulse width of the 5 ns laser pulse). Two digital oscilloscopes (LeCroy 9361 and 9400 A, 25 and 32 kilobytes of buffer memory per channel, respectively) are used to record the traces of transient transmission changes in two overlapping time windows. The maximal digitizing rate is 10 ns per data point. Transient absorption changes are recorded from 10 ns after the laser pulses until full completion of the photo-transformation. At each wavelength, 25 laser pulses are averaged to improve the signal-to-noise ratio. The quasi-logarithmic data compression reduces the initial number of data points per trace (˜50000) to ˜600 points evenly distributed in a log time scale giving ˜100 points per time decade. The wavelengths are varied from 300 to 730 nm in steps of 2 nm (altogether, 216 spectral points) using a computer-controlled step-motor. Absorption spectra of the samples are measured before and after each experiment on standard spectrophotometer (Beckman DU-800).

Each data set is independently analyzed using the global multi-exponential nonlinear least-squares fitting program MEXFIT (Gordeliy, V. I. et al. Molecular basis of transmembrane signalling by sensory rhodopsin II-transducer complex. Nature 419, 484-487 (2002); incorporated herein by reference). The number of exponential components is incremented until the standard deviation of weighted residuals did not further improve. After establishing the apparent rate constants and their assignment to the internal irreversible transitions of a single chain of relaxation processes, the amplitude spectra of exponents are transformed to the difference spectra of the corresponding intermediates in respect to the spectrum of final state. Subsequently, the absolute absorption spectra of states are determined by adding the difference spectra divided by the fraction of converted molecules to the spectra of the final states. Criteria for the determination of the fraction value are the absence of negative absorbencies and contributions from the initial state to the calculated spectra of final state. For further details of the methods see (Chizhov, I. et al. Biophys. J. 71, 2329-2345 (1996)).

In addition, the light-driven inward directed proton pump can be further electrophysiologically characterized by using patch-clamp measurements in the whole cell configuration using an Axopatch 200B interface, Axon Instruments. Photocurrents are measured in response to light pulses with a saturating intensity of 23 mW/mm² using diode-pumped solid-state lasers (λ=532 nm) focused into a 400-μm optic fiber. Light pulses are applied by a fast computer-controlled shutter (Uniblitz LS6ZM2, Vincent Associates). Ultrashort nanosecond light pulses are generated by the Opolette 355 tunable laser system (OPTOPRIM). For the measurement of the actionspectra the pulse energies at the different wavelengths were set to values which corresponded to equal photon counts of 10¹⁹ photons/m². Moreover photocurrent-voltage relationships at membrane potentials ranging from −100 mV to +60 mV were measured (except for On/Off kinetics, where membrane potentials ranged from −80 mV to +80 mV). Patch pipettes with resistances of 2-5 MΩ can be fabricated from thin-walled borosilicate glass (GB150F-8P) on a horizontal puller (Model P-1000, Sutter Instruments). Further guidance is provided in Example 3 below.

Briefly, the candidate light-driven inwardly directed proton pump can be heterologously expressed in rat hippocampal neurons by means of adeno-associated virus mediated gene transfer. Hippocampi are isolated from postnatal P1 Sprague-Dawley rats and treated with papain (20 U ml⁻¹) for 20 min at 37° C. The hippocampi are washed with DMEM (Invitrogen/Gibco, high glucose) supplemented with 10% fetal bovine serum and titrated in a small volume of this solution. ˜96,000 cells are plated on poly-D-lysine/laminin coated glass cover slips in 24-well plates. After 3 hours the plating medium is replaced by culture medium (Neurobasal A containing 2% B-27 supplement, and 2 mM Glutamax-I).

rAAV2/1 virus is prepared using a pAAV2 vector with a human synapsin promoter containing the DNA sequence of the light-driven inwardly directed proton pump, C-terminally fused to the Kir2.1 membrane trafficking signal, a P2A self-cleaving peptide and a GFP variant. Briefly 5×10⁹ genome copies/ml (GC/ml) of rAAV2/1 virus is added to each well 4-9 days after plating. The electrophysiological recordings are performed 19-23 days after transduction.

The electrophysiological characterization is performed using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3. The extracellular solution contains 125 mM NaCl, 2 mM KCl, 2 mM CaCl₂, 1 mM MgCl₂, 30 mM glucose and 25 mM HEPES, titrated to pH 7.3. Electrophysiological signals are filtered at 10 kHz, digitized with an Axon Digidata 1322A (50 kHz) and acquired and analyzed using pClamp9 software (Axon Instruments).

In some embodiments, the light-driven inward directed proton pump of the present disclosure has a turnover rate of more than 250 s⁻¹, preferably more than 300 s⁻¹, more preferably more than 370 s⁻¹, more preferably more than 380 s⁻¹, more preferably more than 390 s⁻¹, such as a turnover rate of 400 s⁻¹, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.

In certain embodiments, the light-driven inward directed proton pump is capable of triggering action potentials in a frequency of more than 40 Hz, preferably in a frequency of more than 50 Hz, more preferably in a frequency of more than 60 Hz, even more preferably in a frequency of more than 70 Hz, and most preferably in a frequency of 80 Hz, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.

Alternatively, or in addition, the light-driven inward directed proton pump is capable of being triggered with a pulse width of 3 ms of λ=532 nm and an intensity of 23 mW/mm², if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.

In a related aspect, the present disclosure also provides a nucleic acid construct, comprising a nucleotide sequence coding for the light-driven inward directed proton pump as described above.

To ensure optimal expression, the coding nucleotide sequence can also be suitably modified, for example by adding suitable regulatory sequences and/or targeting sequences and/or by matching of the coding DNA sequence to the preferred codon usage of the chosen host. In a particularly preferred embodiment, the nucleotide sequence is codon-optimized for expression in human cells. For example, the nucleotide sequence may have the sequence shown in SEQ ID NO: 16. The targeting sequence may encode a C-terminal extension targeting the light-inducible inward proton pump to a particular site or compartment within the cell, such as to the synapse or to a post-synaptic site, to the axon-hillock, or the endoplasmic reticulum. The nucleic acid may be combined with further elements, e.g., a promoter and a transcription start and stop signal and a translation start and stop signal and a polyadenylation signal in order to provide for expression of the sequence of the mutant light-inducible inward proton pump of the present disclosure. The promoter can be inducible or constitutive, general or cell specific promoter. An example of a cell-specific promoter is the mGlu6-promotor specific for bipolar cells. In particular embodiments, the coding sequence of the light-driven inward directed proton pump is under the control of a neuronal cell specific human promotor, preferably the human synapsin promotor. Selection of promoters, vectors and other elements is a matter of routine design within the level of ordinary skill in the art. Many such elements are described in the literature and are available through commercial suppliers.

Also disclosed is an expression vector, comprising the nucleotide sequence coding for the mutant light-inducible inward proton pump or the nucleic acid construct as disclosed herein, wherein the nucleotide sequence is optimized for expression in human cells. In a preferred embodiment, the vector is suitable for gene therapy, in particular wherein the vector is suitable for virus-mediated gene transfer, i.e. wherein the vector is a viral vector. The term “suitable for virus-mediated gene transfer” means herein that said vector can be packed in a virus and thus be delivered to the site or the cells of interest. Examples of viruses suitable for gene therapy are retroviruses, adenoviruses, adeno-associated viruses, lentiviruses, pox viruses, alphaviruses, rabies virus, semliki forest virus and herpes viruses. These viruses differ in how well they transfer genes to the cells they recognize and are able to infect, and whether they alter the cell's DNA permanently or temporarily. However, gene therapy also encompasses non-viral methods, such as application of naked DNA, lipoplexes and polyplexes, and dendrimers.

The resulting nucleic acid sequence may be introduced into cells e.g. using a virus as a carrier or by transfection including e.g. by chemical transfectants (such as Lipofectamine, Fugene, etc.), electroporation, calcium phosphate co-precipitation and direct diffusion of DNA. A method for transfecting a cell is detailed in the examples and may be adapted to the respective recipient cell. Transfection with DNA yields stable cells or cell lines, if the transfected DNA is integrated into the genome, or unstable (transient) cells or cell lines, wherein the transfected DNA exists in an extrachromosomal form. Furthermore, stable cell lines can be obtained by using episomal replicating plasmids, which means that the inheritance of the extrachromosomal plasmid is controlled by control elements that are integrated into the cell genome. In general, the selection of a suitable vector or plasmid depends on the intended host cell.

Therefore, the present disclosure also pertains to a mammalian cell expressing the light-driven inward directed proton pump as disclosed herein, with the proviso that the mammalian cell is not a human embryonic cell or a cell capable of modifying the germ line genetic identity of human beings. Similarly, the present disclosure provides a mammalian cell comprising the nucleic acid construct, or the expression vector a disclosed herein.

The incorporation of the light-driven inward proton pump of the present disclosure into the membrane of cells which do not express the corresponding channels in nature can for example be simply effected in that, using known procedures of recombinant DNA technology, the DNA coding for this inward proton pump is firstly incorporated into a suitable expression vector, e.g. a plasmid, a cosmid or a virus, the target cells are then transformed with this, and the protein is expressed in this host. Next, the cells are treated in a suitable manner, e.g. with retinal, in order to enable the linkage of a Schiff's base between protein and retinal.

The expression of the light-driven inward proton pump of the present disclosure can be advantageously effected in certain mammalian cell systems. The expression is effected either with episomal vectors as transient expression, preferably in neuroblastoma cells (e.g., NG108-15-Cells), melanoma cells (e.g., the BLM cell line), COS cells (generated by infection of “African green monkey kidney CV1” cells) or HEK cells (“human embryonic kidney cells”, e.g. HEK293 cells), or BHK-cells (“baby hamster kidney cells”), or in the form of stable expression (by integration into the genome) in CHO cells (“Chinese hamster ovary cells”), myeloma cells or MDCK cells (“Madine-Darby canine kidney cells”) or in Sf9 insect cells infected with baculoviruses. lin a more preferred embodiment the mammalian cell is a neuroblastoma cell, in particular NG108-15; a HEK293 cell; a COS cell; a BHK cell; a CHO cell; a myeloma cell; or a MDCK cell.

In another preferred embodiment, the mammalian cell is an electrically excitable cell. It is further preferred that the cell is a hippocampal cell, a photoreceptor cell; a retinal rod cell; a retinal cone cell; a retinal ganglion cell; a bipolar neuron; a ganglion cell; a pseudounipolar neuron; a multipolar neuron; a pyramidal neuron, a Purkinje cell; or a granule cell.

A neuron is an electrically excitable cell that processes and transmits information by electrical and chemical signalling, wherein chemical signaling occurs via synapses, specialized connections with other cells. A number of specialized types of neurons exist such as sensory neurons responding to touch, sound, light and numerous other stimuli affecting cells of the sensory organs, motor neurons receiving signals from the brain and spinal cord and causing muscle contractions and affecting glands, and interneurons connecting neurons to other neurons within the same region of the brain or spinal cord. Generally, a neuron possesses a soma, dendrites, and an axon. Dendrites are filaments that arise from the cell body, often extending for hundreds of microns and branching multiple times. An axon is a special cellular filament that arises from the cell body at a site called the axon hillock. The cell body of a neuron frequently gives rise to multiple dendrites, but never to more than one axon, although the axon may branch hundreds of times before it terminates. At the majority of synapses, signals are sent from the axon of one neuron to a dendrite of another. There are, however, many exceptions to these rules: neurons that lack dendrites, neurons that have no axon, synapses that connect an axon to another axon or a dendrite to another dendrite, etc. Most neurons can further be anatomically characterized as unipolar or pseudounipolar (dendrite and axon emerge from same process), bipolar (axon and single dendrite on opposite ends of the soma), multipolar (having more than two dendrites and may be further classified as (i) Golgi I neurons with long-projecting axonal processes, such as pyramidal cells, Purkinje cells, and anterior horn cells, and (ii) Golgi II: neurons whose axonal process projects locally, e.g., granule cells.

A photoreceptor cell, is a specialized neuron found in the retina that is capable of phototransduction. The two classic photoreceptors are rods and cones, each contributing information used by the visual system. A retinal ganglion cell is a type of neuron located near the inner surface of the retina of the eye. These cells have dendrites and long axons projecting to the protectum (midbrain), the suprachiasmatic nucleus in the hypothalamus, and the lateral geniculate (thalamus). A small percentage contribute little or nothing to vision, but are themselves photosensitive. Their axons form the retinohypothalamic tract and contribute to circadian rhythms and pupillary light reflex, the resizing of the pupil. They receive visual information from photoreceptors via two intermediate neuron types: bipolar cells and amacrine cells. Amacrine cells are interneurons in the retina, and responsible for 70% of input to retinal ganglion cells. Bipolar cells, which are responsible for the other 30% of input to retinal ganglia, are regulated by amacrine cells. As a part of the retina, the bipolar cell exists between photoreceptors (rod cells and cone cells) and ganglion cells. They act, directly or indirectly, to transmit signals from the photoreceptors to the ganglion cells.

The cell may be isolated (and genetically modified), maintained and cultured at an appropriate temperature and gas mixture (typically, 37° C., 5% CO2), optionally in a cell incubator as known to the skilled person and as exemplified for certain cell lines or cell types in the examples. Culture conditions may vary for each cell type, and variation of conditions for a particular cell type can result in different phenotypes. Aside from temperature and gas mixture, the most commonly varied factor in cell culture systems is the growth medium. Recipes for growth media can vary in pH, glucose concentration, growth factor and the presence of other nutrient components among others. Growth media are either commercially available, or can be prepared according to compositions, which are obtainable from the American Tissue Culture Collection (ATCC). Growth factors used for supplement media are often derived from animal blood such as calf serum. Additionally, antibiotics may be added to the growth media. Amongst the common manipulations carried out on culture cells are media changes and passaging cells. Thus, the presently disclosed light-driven inward directed proton pump is particularly useful as a research tool, such as in a non-therapeutic use for light-stimulation of electrically excitable cells, in particular neuron cells. Further guidance, e.g., with regard to Hippocampal neuron culture, and electrophysiological recordings from hippocampal neurons, as well as electrophysiological recordings on HEK293 cells, can be found in the examples section herein below.

As an alternative to cells, the present disclosure also provides a liposome, comprising the light-driven inward directed proton pump as disclosed herein and/or as defined in the claims.

In general, the retinal or retinal derivative necessary for the functioning of the light-driven inward proton pump of the present disclosure is produced by the cell to be transfected with said inward proton pump. Depending on its conformation, the retinal may be all-trans retinal, 11-cis-retinal, 13-cis-retinal, or 9-cis-retinal. However, as noted above, it is also contemplated that of the light-driven inward proton pump of the present disclosure may be incorporated into vesicles, liposomes or other artificial cell membranes. Accordingly, also disclosed is a channelrhodopsin, comprising the light-driven inward proton pump of the present disclosure, and a retinal or retinal derivative. Preferably, the retinal derivative is selected from the group consisting of 3,4-dehydroretinal, 13-ethylretinal, 9-dm-retinal, 3-hydroxyretinal, 4-hydroxyretinal, naphthylretinal; 3,7,11-trimethyl-dodeca-2,4,6,8, 10-pentaenal; 3,7-dimethyl-deca-2,4,6,8-tetraenal; 3,7-dimethyl-octa-2,4,6-trienal; and 6-7 rotation-blocked retinals, 8-9 rotation-blocked retinals, and 10-11 rotation-blocked retinals.

Finally, there are a number of diseases in which, e.g., the natural visual cells no longer function, but all nerve connections are capable of continuing to operate. Today, attempts are being made in various research centres to implant thin films with artificial ceramic photocells on the retina. These photocells are intended to depolarise the secondary, still intact cells of the retinal and thereby to trigger a nerve impulse (bionic eyes). The deliberate expression of mutant light-controlled inward proton pumps according to the present disclosure in these ganglion cells, amacrine cells or bipolar cells would be a very much more elegant solution and enable greater three-dimensional visual resolution.

Therefore, the present disclosure also contemplates the light-driven inward proton pump, the nucleic acid construct, the expression vector, the mammalian cell, or the liposome according to the present disclosure for use in medicine.

As shown in the examples below, the proof of principle was already demonstrated in the art, and can easily be adapted to the presently disclosed light-driven inward proton pumps. In view of these data, it is contemplated that the presently disclosed light-inducible inward proton pumps can be used for restoring auditory activity in deaf subjects, or recovery of vision in blind subjects.

Due to its pH-modifying capabilities, the light-driven proton pump may also be used in treating or alleviating alkalosis. Likewise, it is contemplated that due to its electrophysiologically capabilities, the light-driven inward proton pump of the present disclosure can be suitably applied in treating or alleviating neurological injury, brain damage, seizure, or a degenerative neurological disorder, such as Parkinson's disease and Alzheimer's disease. In all these treatment cases, the light-driven inward proton pump may be delivered by way of liposomes, and more preferably by way of administering the nucleic acid construct or the expression vector of the present disclosure to subject to be treated.

Further described are non-human animals which comprise a cell according to the present disclosure, i.e. a cell which functionally express the light-driven inward proton pump according to the present disclosure, e.g. in an cell such as a neuron, in particular in spiral ganglion neurons, as also described for the cell of the present disclosure. In preferred embodiments, the cell is an endogenous cell. The non-human animal may be any animal other than a human. In a preferred embodiment, the non-human animal is a vertebrate, preferably a mammal, more preferably a rodent, such as a mouse or a rat, or a primate.

In particular, some model organisms are preferred, such as Caenorhabditis elegans, Arbacia punctulata, Ciona intestinalis, Drosophila, usually the species Drosophila melanogaster, Euprymna scolopes, Hydra, Loligo pealei, Pristionchus pacificus, Strongylocentrotus purpuratus, Symsagittifera roscoffensis, and Tribolium castaneum. Among vertebrates, these are several rodent species such as guinea pig (Cavia porcellus), hamster, mouse (Mus musculus), and rat (Rattus norvegicus), as well as other species such as chicken (Gallus gallus domesticus), cat (Felis cattus), dog (Canis lupus familiaris), Lamprey, Japanese ricefish (Oryzias latipes), Rhesus macaque, Sigmodon hispidus, zebra finch (Taeniopygia guttata), pufferfish (Takifugu rubripres), african clawed frog (Xenopus laevis), and zebrafish (Danio rerio). Also preferred are non-human primates, i.e. all species of animals under the order Primates that are not a member of the genus Homo, for example rhesus macaque, chimpanzee, baboon, marmoset, and green monkey. However, these examples are not intended to limit the scope of the invention. In any case, it is noted that those animals are excluded, which are not likely to yield in substantial medical benefit to man or animal and which are therefore not subject to patentability under the respective patent law or jurisdiction. Moreover, the skilled person will take appropriate measures, as e.g. laid down in international guidelines of animal welfare, to ensure that the substantial medical benefit to man or animal will outweigh any animal suffering.

Finally, also non-therapeutic, or ex vivo, or in vitro uses of the light-driven inward directed proton pump of the present disclosure are contemplated. For example, the light-driven inward directed proton pump of the present disclosure may be advantageously applied (i) for light-stimulation of electrically excitable cells, (ii) for transporting protons over a membrane against a proton concentration gradient, (iii) for acidifying or alkalinizing the interior of a cell, cell compartment, vesicle, or liposome, or (iv) or as an optogenetic tool.

The present invention is further illustrated by the following embodiments:
1. A light-driven inward directed proton pump having at least 59% sequence similarity over the full length of SEQ ID NO: 1 (NsXeR) for use in medicine.
2. The light-driven inward directed proton pump for use of embodiment 1, wherein the light-driven inward directed proton pump has at least 65%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably 99% sequence similarity to the full length of SEQ ID NO: 1 (NsXeR); and/or
wherein the light-driven inward directed proton pump has at least 38%, more preferably at least 45%, more preferably at least 48%, more preferably at least 50%, more preferably at least 55%, more preferably at least 56%, more preferably at least 57%, more preferably at least 58%, more preferably at least 59%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% sequence identity to the full length of SEQ ID NO: 1 (NsXeR).
3. The light-driven inward directed proton pump for use of embodiment 1 or 2, wherein light-driven inward directed proton pump is not mutated at position E4, H48, S55, W73, D76, S80, A87, P209, C212, K214, and D220 of SEQ ID NO: 1.
4. The light-driven inward directed proton pump for use of any one of embodiments 1-3, wherein light-driven inward directed proton pump is not truncated at the N-terminus.
5. The light-driven inward directed proton pump for use of embodiment 1, wherein the light-driven inward directed proton pump comprises an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5); in particular wherein the light-driven inward directed proton pump comprises the amino acid sequence of SEQ ID NO: 1 (NsXeR).
6. The light-driven inward directed proton pump for use of embodiment 1, wherein the light-driven inward directed proton pump consists of an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5); in particular wherein the light-driven inward directed proton pump consists of the amino acid sequence of SEQ ID NO: 1 (NsXeR).
7. The light-driven inward directed proton pump for use of any one of embodiments 1-6, wherein the light-driven inward directed proton pump is active between pH 6 and pH 8; preferably between pH 5 and pH 9.
8. The light-driven inward directed proton pump for use of any one of embodiments 1-7, wherein the absorption maximum of the light-driven inward directed proton pump is between 560 nm and 580 nm.
9. The light-driven inward directed proton pump for use of any one of embodiments 1-8, wherein the photocycle of the light-driven inward directed proton pump is less than 50 ms, preferably less than 45 ms, more preferably less than 40 ms, more preferably less than 35 ms, even more preferably less than 30 ms, such as 27 ms, if measured in proteo-nanodiscs exhibiting a molar ratio of DMPC:MSP1E3:light-driven inward directed proton pump of 100:2:3 at 20° C. and pH 7.5, providing pulses of 5 ns duration at 532 nm wavelength and energy of 3 mJ/pulse.
10. The light-driven inward directed proton pump for use of any one of embodiments 1-9, wherein the light-driven inward directed proton pump has a turnover rate of more than 250 s⁻¹, preferably more than 300 s⁻¹, more preferably more than 370 s⁻¹, more preferably more than 380 s⁻¹, more preferably more than 390 s⁻¹, such as a turnover rate of 400 s⁻¹, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.
11. The light-driven inward directed proton pump for use of any one of embodiments 1-10, wherein the light-driven inward directed proton pump is capable of triggering action potentials in a frequency of more than 40 Hz, preferably in a frequency of more than 50 Hz, more preferably in a frequency of more than 60 Hz, even more preferably in a frequency of more than 70 Hz, and most preferably in a frequency of 80 Hz, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.
12. The light-driven inward directed proton pump for use of any one of embodiments 1-11, wherein the light-driven inward directed proton pump is capable of being triggered with a pulse width of 3 ms of λ=532 nm and an intensity of 23 mW/mm², if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.
13. A nucleic acid construct, comprising a nucleotide sequence coding for the light-driven inward directed proton pump as defined in any one of embodiments 1-12, wherein the nucleotide sequence is codon-optimized for expression in human cells; preferably wherein the nucleotide sequence has the sequence shown in SEQ ID NO: 16.
14. An expression vector, comprising a nucleotide sequence coding for light-driven inward directed proton pump as defined in any one of embodiments 1-12 or the nucleic acid construct according to embodiment 13, wherein the nucleotide sequence is optimized for expression in human cells.
15. The expression vector of embodiment 14, wherein the vector is a viral vector.
16. The expression vector of embodiment 14 or 15, wherein the coding sequence of the light-driven inward directed proton pump is under the control of a neuronal cell specific human promotor, preferably the human synapsin promotor.
17. A mammalian cell expressing the light-driven inward directed proton pump as defined in any one of embodiments 1-12, with the proviso that the mammalian cell is not a human embryonic cell or a cell capable of modifying the germ line genetic identity of human beings.
18. A mammalian cell comprising the nucleic acid construct according to embodiment 13 or the expression vector according to any one of embodiments 14-16.
19. The mammalian cell of embodiment 17 or 18, wherein the cell is
   i. a hippocampal cell, a photoreceptor cell, a retinal rod cell, a retinal cone cell, a retinal ganglion cell, a bipolar neuron, a ganglion cell, a pseudounipolar neuron, a multipolar neuron, a pyramidal neuron, a Purkinje cell, or a granule cell; or
   ii. a neuroblastoma cell, in particular NG108-15; a HEK293 cell; a COS cell; a BHK cell; a CHO cell; a myeloma cell; or a MDCK cell.
20. A liposome, comprising the light-driven inward directed proton pump as defined in any one of embodiments 1-12.
21. The nucleic acid construct according to embodiment 13, the expression vector according to any one of embodiments 14-16, the mammalian cell according to any one of embodiments 17-19, or the liposome according to embodiment 20 for use in medicine.
22. The light-driven inward directed proton pump as defined in any one of embodiments 1-12, the nucleic acid construct according to embodiment 13, the expression vector according to any one of embodiments 14-16, the mammalian cell according to any one of embodiments 17-19, or the liposome according to embodiment 20 for use in restoring auditory activity, recovery of vision, or for use in treating or alleviating alkalosis, neurological injury, brain damage, seizure, or a degenerative neurological disorder, such as Parkinson's disease and Alzheimer's disease.
23. A non-human mammal, comprising a cell according to any one of embodiments 17-19, preferably wherein the cell is an endogenous cell; with the proviso that those animals are excluded, which are not likely to yield in substantial medical benefit to man or animal which will outweigh any animal suffering.
24. A non-therapeutic, or ex vivo, or in vitro use of a light-driven inward directed proton pump as defined in any one of embodiments 1-12,
   (i) for light-stimulation of electrically excitable cells,
   (ii) for transporting protons over a membrane against a proton concentration gradient,
   (iii) for acidifying or alkalinizing the interior of a cell, cell compartment, vesicle, or liposome, or
   (iv) or as an optogenetic tool.

In the following, the present invention is illustrated by figures and examples which are not intended to limit the scope of the present invention.

### DESCRIPTION OF THE FIGURES

FIG. 1: Sequence alignment of microbial rhodopsins. The sequence alignment was performed with Clustal Omega. Helices regions are marked with “+” sign, N-terminal and transmembrane helices are subscribed. The motif amino acids and the H48-D220 proton acceptor pair are highlighted in bold.

Specification of UniProtIDs for the sequences. NsXeR (G0QG75), HrvXeR1 (H0AAK5), ASR (Q8YSC4), HsBR (P02945), PR (Q9F7P4), NpHR (P15647), DeKR2 (N0DKS8), NpSR2 (P42196), HrvXeR (Ghai et al., supra), AlkXeR (Vavourakis et al., supra), AlkXeRs1-5 (Vavourakis et al., supra).

FIG. 2: Electrogenic properties of XeR. a. pH changes upon illumination in E. coli cell suspensions expressing different XeRs. Graphs show the pH changes with and without the addition of CCCP. b. pH changes upon illumination in liposome suspension with reconstructed NsXeR (with and without CCCP). c. pH changes upon illumination in liposomes suspension measured under different pH values.

FIG. 3: Spectroscopic characterization of NsXeR. a. Absorption spectra of representatives of xenorhodopsin family solubilized in the detergent DDM. The corresponding positions of absorption maximum is indicated in the legend. b. Transient absorption changes of NsXeR (pH 7.5, T=20° C.) at three characteristic wavelengths 378, 408, and 564 nm. Black lines are experimental data and light gray and dark gray lines represent the result of global fit using five exponents. The photocycles were measured for the two preparations: NsXeR in nanodiscs (light gray) and in liposomes (dark gray). Note that the differences in amplitudes between the samples are due to the approximately two times higher concentration of NsXeR in liposomes than in nanodiscs (see FIG. 4). c. Proposed model of NsXeR photocycle in nanodiscs.

FIG. 4: Photocycles of the NsXeR in nanodiscs (ND, upper row) and liposomes (LIP, lower row) preparations (20° C., pH 7.5). Five kinetically distinct protein states (red lines) are obtained via global multi exponential analysis of the flash photolysis data exemplified in the FIG. 3b. An each panel contains for the reference the correspondent spectrum on unexcited protein (P₀, black lines). The spectra of P_{i=1 . . . 5} states were calculated from correspondent spectra of exponents, which were further converted to the differential spectra of the states assuming the sequential irreversible model of the photocycle. The half-times of reactions are depicted between the panels. The fraction of cycled molecules was 12.5% in ND, and 15% in LIP.

FIG. 5: Photocurrents in HEK293 and NG108-15 cells. Photocurrents in cells expressing NsXeR at the membrane potentials changed in 20 mV steps from −100 mV and corresponding I-V curves. a. HEK293 with pipette solution 1 and bath solution 1. b. NG108-15 cells with pipette solution 2 and bath solution 2 (control measurements to confirm that protons are responsible for inwardly directed current).

FIG. 6: Spiking traces at different light-pulse frequencies. Rat hippocampal neurons heterologously expressing NsXeR were investigated by patch-clamp experiments in the whole cell configuration under current clamp conditions. Action potentials were triggered by 40 light-pulses at indicated frequencies. The light pulses had a pulse width of 3 ms, a wavelength of λ=532 nm and an intensity of 23 mW/mm².

FIG. 7: Variability of spike latency. Exemplary spiking traces measured in different neuronal cells. The light pulses had a pulse width of A) 3 ms and B) 10 ms. Rat hippocampal neurons heterologously expressing NsXeR were investigated by patch-clamp experiments in the whole cell configuration under current clamp conditions. The spikes were triggered by light pulses with a wavelength of λ=532 nm and an intensity of 23 mW/mm².

FIG. 8: On and Off kinetics of NsXeR measured in NG108 cells at indicated membrane potentials. Ultrashort nanosecond light pulses were generated by the Opolette 355 at the wavelength of λ=570 nm. Corresponding I-V curve is shown on the right, peak photocurrent is plotted against membrane potential.

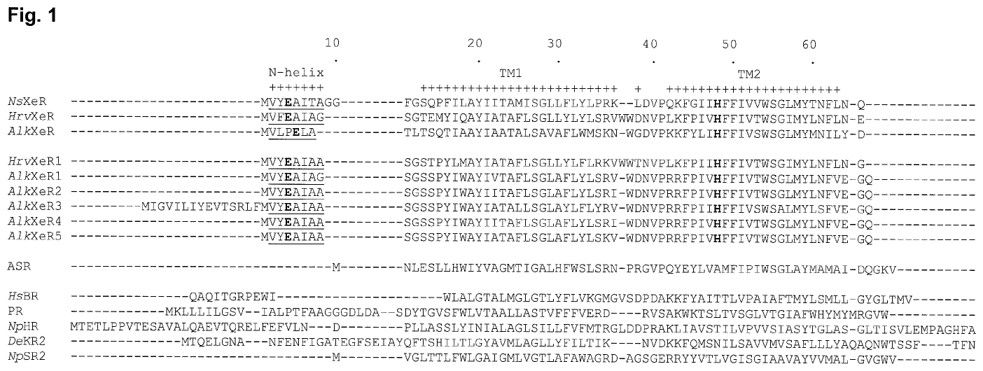

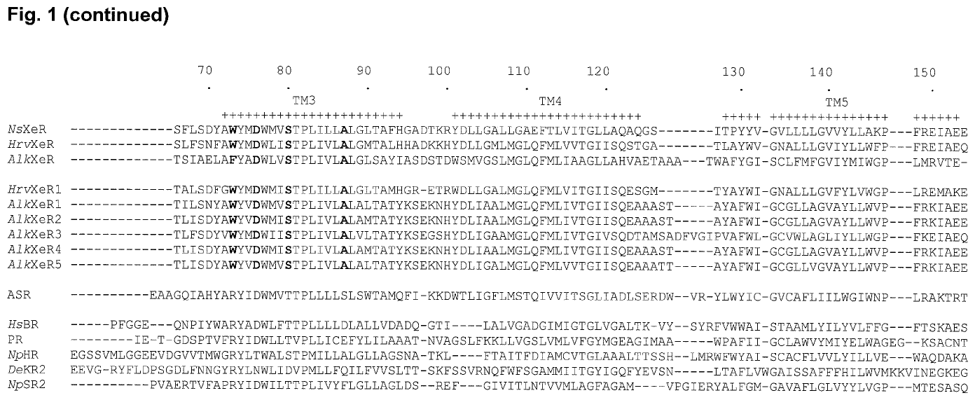

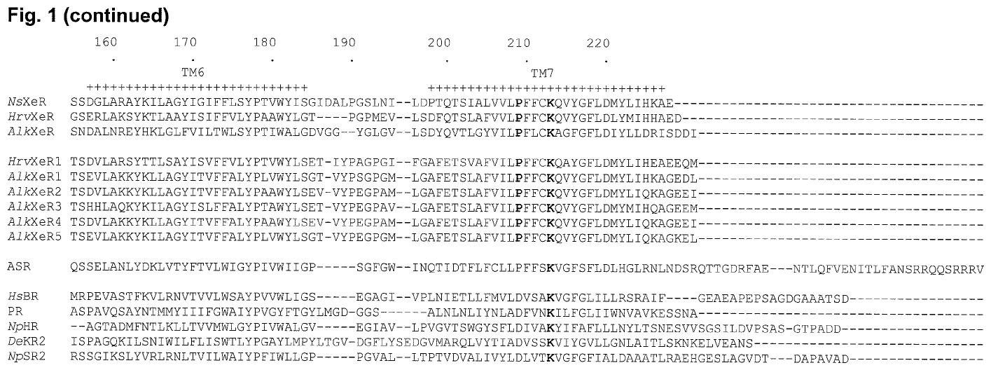

### DESCRIPTION OF THE SEQUENCES

SEQ ID NO: 1 (NsXeR; UniProtID G0QG75, N-terminal helix underlined; motif
amino acids and H48-D220 proton acceptor pair in bold)
MVYEAITAGG FGSQPFILAY IITAMISGLL FLYLPRKLDV PQKFGIIHFF
IVVWSGLMYT NFLNQSFLSD YAWYMDWMVS TPLILLALGL TAFHGADTKR
YDLLGALLGA EFTLVITGLL AQAQGSITPY YVGVLLLLGV VYLLAKPFRE
IAEESSDGLA RAYKILAGYI GIFFLSYPTV WYISGIDALP GSLNILDPTQ
TSIALVVLPF FCKQVYGFLD MYLIHKAE
SEQ ID NO: 2 (HrvXeR1; UniProtID H0AAK5, N-terminal helix underlined; motif
amino acids and H48-D220 proton acceptor pair in bold)
MVYEAIAASG STPYLMAYIA TAFLSGLLYL FLRKVWWTNV PLKFPIIHFF
IVTWSGIMYL NFLNGTALSD FGWYMDWMIS TPLILLALGL TAMHGRETRW
DLLGALMGLQ FMLVITGIIS QESGMTYAYW IGNALLLGVF YLVWGPLREM
AKETSDVLAR SYTTLSAYIS VFFVLYPTVW YLSETIYPAG PGIFGAFETS
VAFVILPFFC KQAYGFLDMY LIHEAEEQM
SEQ ID NO: 3 (ASR; UniProtID Q8YSC4)
MNLESLLHWI YVAGMTIGAL HFWSLSRNPR GVPQYEYLVA MFIPIWSGLA
YMAMAIDQGK VEAAGQIAHY ARYIDWMVTT PLLLLSLSWT AMQFIKKDWT
LIGFLMSTQI VVITSGLIAD LSERDWVRYL WYICGVCAFL IILWGIWNPL
RAKTRTQSSE LANLYDKLVT YFTVLWIGYP IVWIIGPSGF GWINQTIDTF
LFCLLPFFSK VGFSFLDLHG LRNLNDSRQT TGDRFAENTL QFVENITLFA
NSRRQQSRRR V
SEQ ID NO: 4 (HsBR; UniPotID P02945)
MLELLPTAVE GVSQAQITGR PEWIWLALGT ALMGLGTLYF LVKGMGVSDP
DAKKFYAITT LVPAIAFTMY LSMLLGYGLT MVPFGGEQNP IYWARYADWL
FTTPLLLLDL ALLVDADQGT ILALVGADGI MIGTGLVGAL TKVYSYRFVW
WAISTAAMLY ILYVLFFGFT SKAESMRPEV ASTFKVLRNV TVVLWSAYPV
VWLIGSEGAG IVPLNIETLL FMVLDVSAKV GFGLILLRSR AIFGEAEAPE
PSAGDGAAAT SD
SEQ ID NO: 5 (PR; UniProtID: Q9F7P4)
MKLLLILGSV IALPTFAAGG GDLDASDYTG VSFWLVTAAL LASTVFFFVE
RDRVSAKWKT SLTVSGLVTG IAFWHYMYMR GVWIETGDSP TVFRYIDWLL
TVPLLICEFY LILAAATNVA GSLFKKLLVG SLVMLVFGYM GEAGIMAAWP
AFIIGCLAWV YMIYELWAGE GKSACNTASP AVQSAYNTMM YIIIFGWAIY
PVGYFTGYLM GDGGSALNLN LIYNLADFVN KILFGLIIWN VAVKESSNA
SEQ ID NO: 6 (NpHR; UniProtID P15647)
MTETLPPVTE SAVALQAEVT QRELFEFVLN DPLLASSLYI NIALAGLSIL
LFVFMTRGLD DPRAKLIAVS TILVPVVSIA SYTGLASGLT ISVLEMPAGH
FAEGSSVMLG GEEVDGVVTM WGRYLTWALS TPMILLALGL LAGSNATKLF
TAITFDIAMC VTGLAAALTT SSHLMRWFWY AISCACFLVV LYILLVEWAQ
DAKAAGTADM FNTLKLLTVV MWLGYPIVWA LGVEGIAVLP VGVTSWGYSF
LDIVAKYIFA FLLLNYLTSN ESVVSGSILD VPSASGTPAD D
SEQ ID NO: 7 (DeKR2; UniProtID N0DKS8)
MTQELGNANF ENFIGATEGF SEIAYQFTSH ILTLGYAVML AGLLYFILTI
KNVDKKFQMS NILSAVVMVS AFLLLYAQAQ NWTSSFTFNE EVGRYFLDPS
GDLFNNGYRY LNWLIDVPML LFQILFVVSL TTSKFSSVRN QFWFSGAMMI
ITGYIGQFYE VSNLTAFLVW GAISSAFFFH ILWVMKKVIN EGKEGISPAG
QKILSNIWIL FLISWTLYPG AYLMPYLTGV DGFLYSEDGV MARQLVYTIA
DVSSKVIYGV LLGNLAITLS KNKELVEANS
SEQ ID NO: 8 (NpSR2; UniProtID P42196)
MVGLTTLFWL GAIGMLVGTL AFAWAGRDAG SGERRYYVTL VGISGIAAVA
YVVMALGVGW VPVAERTVFA PRYIDWILTT PLIVYFLGLL AGLDSREFGI
VITLNTVVML AGFAGAMVPG IERYALFGMG AVAFLGLVYY LVGPMTESAS
QRSSGIKSLY VRLRNLTVIL WAIYPFIWLL GPPGVALLTP TVDVALIVYL
DLVTKVGFGF IALDAAATLR AEHGESLAGV DTDAPAVAD
SEQ ID NO; 9 (HrvXeR, N-terminal helix underlined; motif amino acids and H48-
D220 proton acceptor pair in bold)
MVFEAIAGSGTEMYIQAYIATAFLSGLLYLYLSRVWWDNVPLKFPIVHFFIVTWSGIMYLN
FLNESLFSNFAWYMDWLISTPLIVLALGMTALHHADKKHYDLLGMLMGLQFMLVVTGIISQ
STGATLAYWVGNALLLGVIYLLWFPFREIAEQGSERLAKSYKTLAAYISIFFVLYPAAWYL
GTPGPMEVLSDFQTSLAFVVLPFFCKQVYGFLDLYMIHHAED
SEQ ID NO: 10 (AlkXeR, N-terminal helix underlined; motif amino acids and H48-
D220 proton acceptor pair in bold)
MVLPELATLTSQTIAAYIAATALSAVAFLWMSKNWGDVPKKFYLIHFFIVSWSGLMYMNIL
YDTSIAELAFYADWLVSTPLIVLALGLSAYIASDSTDWSMVGSLMGLQFMLIAAGLLAHVA
ETAAATWAFYGISCLFMFGVIYMIWGPLMRVTESNDALNREYHKLGLFVILTWLSYPTIWA
LGDVGGYGLGVLSDYQVTLGYVILPFLCKAGFGFLDIYLLDRISDDI
SEQ ID NO: 11 (AlkXeR1, N-terminal helix underlined; motif amino acids and H48-
D220 proton acceptor pair in bold)
MVYEAIAGSGSSPYTWAYIVTAFLSGLAFLYLSRVWDNVPRRFPIVHFFIVTWSGLMYLNF
VEGQTILSNYAWYVDWMVSTPLIVLALALTATYKSEKNHYDLIAALMGLQFMLIVTGIISQ
EAAASTAYAFWIGCGLLAGVAYLLWVPFRKIAEETSEVLAKKYKLLAGYITVFFALYPLVW
YLSGTVYPSGPGMLGAFETSLAFVILPFFCKQVYGFLDMYLIHKAGEDL
SEQ ID NO: 12 (AlkXeR2, N-terminal helix underlined; motif amino acids and H48-
D220 proton acceptor pair in bold)
MVYEAIAASGSSPYIWAYIITAFLSGLAFLYLSRIWDNVPRRFPIVHFFIVTWSGLMYLNF
VEGQTLISDYAWYVDWMISTPLIVLALAMTATYKSEKNHYDLIAALMGLQFMLIVTGIISQ
EAAASTAYAFWIGCGLLAGVAYLLWVPFRKIAEETSDVLAKKYKLLAGYITVFFALYPAAW
YLSEVVYPEGPAMLGAFETSLAFVILPFFCKQVYGFLDMYLIQKAGEEI
SEQ ID NO: 13 (AlkXeR3, N-terminal helix underlined; motif amino acids and H48-
D220 proton acceptor pair in bold)
MIGVILIYEVTSRLFMVYEAIAASGSSPYIWAYIATALLSGLAYLFLYRVWDNVPRRFPII
HFFIVSWSALMYLSFVEGQTLFSDYVWYMDWIISTPLIVLALVLTATYKSEGSHYDLIGAA
MGLQFMLIVTGIVSQDTAMSADFVGIPVAFWLGCVWLAGLIYLLWGPFKEIAEQTSHHLAQ
KYKILAGYISLFFALYPTAWYLSETVYPEGPAVLGAFETSLAFVILPFFCKQVYGFLDMYM
IHQAGEEM
SEQ ID NO: 14 (AlkXeR4, N-terminal helix underlined; motif amino acids and H48-
D220 proton acceptor pair in bold)
MVYEAIAASGSSPYIWAYIITAFLSGLAFLYLSRIWDNVPRRFPIVHFFIVTWSGLMYLNF
VEGQTLISDYAWYVDWMISTPLIVLALAMTATYKSEKNHYDLIAALMGLQFMLIVTGIISQ
EAAASTAYAFWIGCGLLAGVAYLLWVPFRKIAEETSDVLAKKYKLLAGYITVFFALYPAAW
YLSEVVYPEGPAMLGAFETSLAFVILPFFCKQVYGFLDMYLIQKAGEEI
SEQ ID NO: 15 (AlkXeR5, N-terminal helix underlined; motif amino acids and H48-
D220 proton acceptor pair in bold)
MVYEAIAASGSSPYIWAYIATAFLSGLAFLYLSKVWDNVPRRFPIVHFFIVTWSGLMYLNF
VEGQTLISDYAWYVDWMVSTPLIVLALALTATYKSEKNHYDLIGALMGLQFMLVVTGIISQ
EAAATTAYAFWIGCGLLVGVAYLLWVPFRKIAEETSEVLAKKYKILAGYITVFFALYPLVW
YLSGTVYPEGPGMLGAFETSLAFVILPFECKQVYGELDMYLIQKAGKEL
SEQ ID NO: 16 (human codon-optimized NsXeR)

| | |
|---|---|
| atggtgtacg aggccatcac agccggcgga ttcggcagcc agcctttcat cctggcctac | 60 |
| atcatcaccg ccatgatcag cggcctgctg ttcctgtacc tgccccggaa gctggacgtg | 120 |
| ccccagaagt tcggcatcat ccactttttc atcgtcgtgt ggagcggcct gatgtatacc | 180 |
| aacttcctga accagagctt cctgagcgac tacgcctggt acatggactg gatggtgtcc | 240 |
| acccccctga tcctgctggc cctgggactg acagctttcc acggcgccga caccaagaga | 300 |
| tacgacctgc tgggagcact gctgggcgcc gagtttaccc tcgtgatcac tggactgctg | 360 |
| gctcaggccc agggctccat caccccttac tatgtgggcg tgctcctgct gctgggggtg | 420 |
| gtgtatctgc tggccaagcc cttcagagag atcgccgagg aaagcagcga cggcctggcc | 480 |
| agagcctaca agatcctggc cggctatatc ggcatcttct ttctgtccta ccccaccgtg | 540 |
| tggtacatca gcggcatcga cgccctgccc ggcagcctga atatcctgga ccctacccag | 600 |
| acctctatcg ccctggtggt gctgccattc ttctgtaaac aagtgtacgg cttcctggac | 660 |
| atgtacctga tccacaaggc tgag | 684 |

### EXAMPLES

### Example 1—Characterization of Xenorhodopsins

pH Changes in E. coli Suspensions

NsXeR (Uniprot ID G0QG75), HrvXeR (Ghai, R. et al. Sci. Rep. 1, (2011)) and AlkXeR (Vavourakis, C. D. et al. Front. Microbiol. 7, (2016)), coding DNAs were synthesized commercially (Eurofins). The nucleotide sequences were optimized for E. coli expression using the GeneOptimizer™ software (Life Technologies, USA). The genes together with the 5′ ribosome-binding sites and the 3′ extensions coding additional LEHHHHHH* tags were introduced into the pET15b expression vector (Novagen) via XbaI and BamHI restriction sites.

The protein was expressed as described previously (Gushchin, I. et al. Crystal structure of a light-driven sodium pump. Nat. Struct. Mol. Biol. 22, 390-395 (2015); incorporated herein in its entirety by reference) with modifications. E. coli cells of strain C41(DE3) (Lucigen) were transformed with the expression plasmids. Transformed cells were grown at 37° C. in shaking baffled flasks in an autoinducing medium, ZYP-5052 (Studier, F. W. Protein production by auto-induction in high-density shaking cultures. Protein Expr. Purif. 41, 207-234 (2005); incorporated herein in its entirety by reference) containing 100 mg/L ampicillin, and were induced at optical density OD600 of 0.6-0.7 with 1 mM isopropyl β-d-1-thiogalactopyranoside (IPTG) and supplemented with 10 μM all-trans-retinal. Three hours after induction, the cells were collected by centrifugation at 3,000 g for 10 min and were washed three times with an unbuffered salt solution (100 mM NaCl, and 10 mM MgCl2) with 30-min intervals between the washes to allow exchange of the ions inside the cells with the bulk. After that, the cells were resuspended in 100 mM NaCl solution and adjusted to an OD600 of 8.5. The measurements were performed in 3 ml aliquots of stirred cell suspension kept at 1° C. The cells were illuminated for 5 min with a halogen lamp (Intralux 5000-1, VOLPI) and the light-induced pH changes were monitored with a pH meter (LAB 850, Schott Instruments).

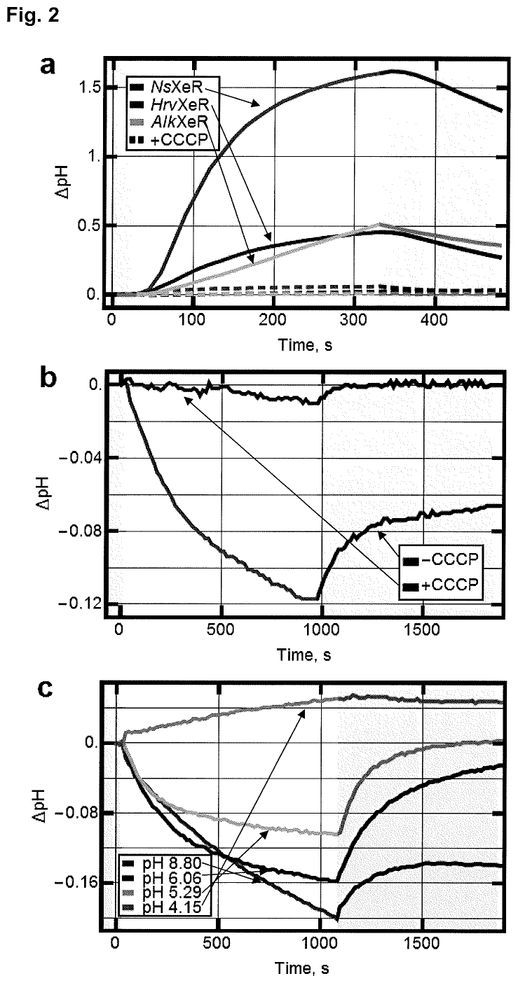

The pH of the cell suspension increased upon illumination and decreased back when the light was turned off (FIG. 2a). The effect of the pH change was completely abolished when repeated under the same conditions after addition of 30 μM of carbonylcyanide m-chlorophenylhydrazone (CCCP). Similar experiments previously done with other proton pumps such as bacteriorhodopsine (BR) gave the opposite behavior of pH upon illumination of the cells (data not shown). Two other members of the xenorhodopsin family, HrvXeR and AlkXeR, studied in the present work gave the same results as NsXeR (FIG. 2a). Thus, pH experiments provide evidence that Nanohaloarchaea rhodopsins are inwardly directed proton pumps.

pH Changes in Liposome Suspension

The protein was expressed as described above. However, three hours after induction, the cells were collected by centrifugation at 3,000 g for 30 min. The collected cells were disrupted in M-110P Lab Homogenizer (Microfluidics, USA) at 25,000 psi in a buffer containing 20 mM Tris-HCl pH 8.0, 5% glycerol, 0.5% Triton X-100 (Sigma-Aldrich, USA) and 50 mg/L DNase I (Sigma-Aldrich, USA). The membrane fraction of cell lysate was isolated by ultracentrifugation at 90,000 g for 1 h at 4° C. The pellet was resuspended in a buffer containing 50 mM NaH₂PO₄/Na₂HPO₄ pH 8.0, 0.1 M NaCl and 1% DDM (Anatrace, Affymetrix, USA) and stirred overnight for solubilization. The insoluble fraction was removed by ultracentrifugation at 90,000 g for 1 h at 4° C. The supernatant was loaded on Ni-NTA column (Qiagen, Germany) and xenorhodopsins were eluted in a buffer containing 50 mM NaH₂PO₄/Na₂HPO₄ pH 7.5, 0.1 M NaCl, 0.3 M imidazole and 0.2% DDM. The eluate was dialysed against 100 volumes of 50 mM NaH₂PO₄/Na₂HPO₄ pH 7.5, 0.1 M NaCl buffer twice for 2 hours to dispose imidazole.

Purified NsXeR were reconstituted in soybean liposomes as described previously (Huang, K. S., Bayley, H. & Khorana, H. G. Delipidation of bacteriorhodopsin and reconstitution with exogenous phospholipid. Proc. Natl. Acad. Sci. 77, 323-327 (1980); incorporated herein by reference). Briefly, phospholipids (asolectin from soybean, Sigma-Aldrich) were dissolved in CHCl₃ (Chloroform ultrapure, Applichem Panreac) and dried under a stream of N₂ in a glass vial. Residual solvent was removed with a vacuum pump overnight. The dried lipids were resuspended at a final concentration of 1% (w/v) in 0.15 M NaCl supplemented with 2% (w/v) sodium cholate. The mixture was clarified by sonication at 4° C. and xenorhodopsin was added at a protein/lipid ratio of 7:100 (w/w). The detergent was removed by overnight stirring with detergent-absorbing beads (Amberlite XAD 2, Supelco). The mixture was dialyzed against 0.15 M NaCl (adjusted to a desired pH) at 4° C. for 1 day (four 200 ml changes) to obtain certain pH.

The measurements were performed on 2 ml of stirred proteoliposome suspension at 0° C. Proteoliposomes were illuminated for 18 minutes with a halogen lamp (Intralux 5000-1, VOLPI) and then were kept in the dark for another 18 minutes. Changes in pH were monitored with a pH meter (LAB 850, Schott Instruments). Measurements were repeated for different starting pH and in the presence of 40 uM of CCCP under the same conditions.

The pH changes upon illumination showed acidification of the solution outside the membrane (FIG. 2b). These pH changes were abolished, when CCCP was added to the suspension. Since in similar experiments all the known outwardly directed proton pumps (like BR and PR) show the opposite pH behavior (Racker, E. & Stoeckenius, W. Reconstitution of purple membrane vesicles catalyzing light-driven proton uptake and adenosine triphosphate formation. J. Biol. Chem. 249, 662-663 (1974)), we conclude that NsXeR are real inwardly directed proton pumps. Interesting is that in a wide range of pH values (between pH 5 and 9) our experiments still show inward proton pumping (FIG. 2c).

Absorption Spectra and Photocycle

Here we report results of the analysis of 2 data sets: the XeR protein reconstituted in nanodiscs and liposomes. The proteo-nanodiscs were assembled using standard protocol (Ritchie, T. K. et al. in Methods in Enzymology (ed. Düzgünes, N.) 464, 211-231 (Academic Press, 2009); incorporated herein by reference). 1,2-dimyristoyl-sn-glycero-3-phosphocholine, DMPC (Avanti Polar Lipids, USA) was used as lipid. An elongated MSP1E3 version of apolipoprotein-1 was used. The molar ratio during assembly was DMPC:MSP1E3:NsXeR=100:2:3. Liposomes were prepared as described above.

The absorption spectra were recorded using the Shimadzu UV-2401PC spectrophotometer. The laser flash photolysis setup was similar to that described by Chizhov and co-workers (Chizhov, I. et al. Spectrally silent transitions in the bacteriorhodopsin photocycle. Biophys. J. 71, 2329-2345 (1996); incorporated herein by reference). The excitation/detection systems were composed as such: a Surelite II-10 Nd:YAG laser (Continuum Inc, USA) was used providing pulses of 5 ns duration at 532 nm wavelength and energy of 3 mJ/pulse. Samples (5×5 mm spectroscopic quartz cuvette (Hellma GmbH & Co, Germany)) were placed in a thermostated house between two collimated and mechanically coupled monochromators (⅛ m model 77250, Oriel Corp., USA). The probing light (Xe-arc lamp, 75 W, Osram, Germany) passed the first monochromator, sample and arrived after a second monochromator at a PMT detector (R3896, Hamamatzu, Japan). The current-to-voltage converter of the PMT determines the time resolution of the measurement system of ca 50 ns (measured as an apparent pulse width of the 5 ns laser pulse). Two digital oscilloscopes (LeCroy 9361 and 9400A, 25 and 32 kilobytes of buffer memory per channel, respectively) were used to record the traces of transient transmission changes in two overlapping time windows. The maximal digitizing rate was 10 ns per data point. Transient absorption changes were recorded from 10 ns after the laser pulses until full completion of the photo-transformation. At each wavelength, 25 laser pulses were averaged to improve the signal-to-noise ratio. The quasi-logarithmic data compression reduced the initial number of data points per trace (˜50000) to ˜600 points evenly distributed in a log time scale giving ˜100 points per time decade. The wavelengths were varied from 300 to 730 nm in steps of 2 nm (altogether, 216 spectral points) using a computer-controlled step-motor. Absorption spectra of the samples were measured before and after each experiment on standard spectrophotometer (Beckman DU-800).

Each data set was independently analyzed using the global multi-exponential nonlinear least-squares fitting program MEXFIT (Gordeliy, V. I. et al. Molecular basis of transmembrane signalling by sensory rhodopsin II-transducer complex. Nature 419, 484-487 (2002); incorporated herein by reference). The number of exponential components was incremented until the standard deviation of weighted residuals did not further improve. After establishing the apparent rate constants and their assignment to the internal irreversible transitions of a single chain of relaxation processes, the amplitude spectra of exponents were transformed to the difference spectra of the corresponding intermediates in respect to the spectrum of final state. Subsequently, the absolute absorption spectra of states were determined by adding the difference spectra divided by the fraction of converted molecules to the spectra of the final states. Criteria for the determination of the fraction value were the absence of negative absorbencies and contributions from the initial state to the calculated spectra of final state. For further details of the methods see (Chizhov, I. et al. Biophys. J. 71, 2329-2345 (1996)).

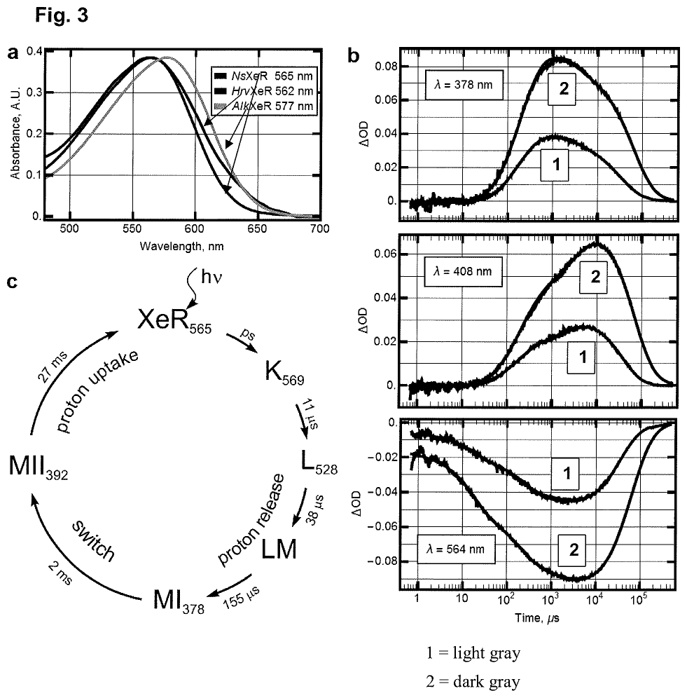

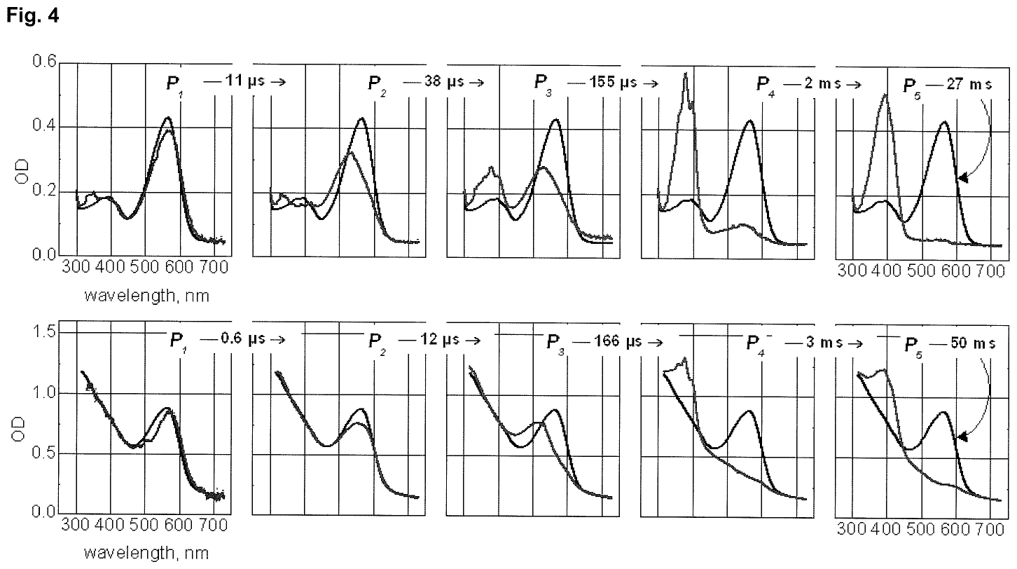

The absorption maximum of NsXeR in solubilized form is 565 nm (FIG. 3a). Its position does not shift when the pH of the buffer is varied in the range from 4.5 to 9.0. NsXeR does not exhibit light and dark adaptation. The homologue AlkXeR is a red-shifted variant, its absorption maximum is 577 nm (FIG. 3a). Transient absorption changes of NsXeR (pH 7.5, T=20° C.) are shown at three characteristic wavelengths 378, 408, and 564 nm with NsXeR prepared in two different ways: in nanodiscs (light gray) and in single lipid vesicles (dark gray) (FIG. 3b). The results of global fit using five exponents are shown in FIG. 4. The photocycle of NsXeR in nanodiscs is faster (27 ms) than in lipid vesicles (50 ms). The photocycle of NsXeR in nanodiscs is shown in FIG. 3c.

The photocycle of NsXeR contains a microsecond part, which is usually assigned to the multistep reaction of a release of the energized ion (the H⁺ in our case) and a millisecond part of relaxation and re-uptake of the ion.

However, the NsXeR photocycle reveals some distinct features, which to our knowledge have never been reported in the previous studies of retinal proteins (FIG. 4). After the microsecond part of the photocycle (P₁, P₂, P₃) that includes archetypical intermediates with the K and L-like spectral shifts (P₁, λₘₐₓ=570 nm, P₂, λₘₐₓ=530 nm), in the millisecond time domain we obtained two spectrally and kinetically different M intermediates (P₄ and P₅). The first M-form (P₄) has a characteristic three-band absorption spectrum with the maximal at 360, 378 and 398 nm. This state with the half-time of 2 in nanodiscs (3 in lipid vesicles) milliseconds converts to the state P₅ with a single maximum at 392 nm. Both intermediates should correspond to the de-protonated state of the retinal Schiff-base. It is interesting that the state P₄ has the same spectral features as a previously reported” spectrum of retro-retinal in bacteriorhodopsin (BR). This form of retinal is characterized by conversion of the backbone carbon C₁₄ from the CH to the CH₂ form with the corresponding change of the C₁₄=C₁₃ double bond to a single one and the alteration of the π-electron conjugation along the retinal. It was reported that the retro-retinal form of BR was achieved by deep UV illumination of the sample and (or) addition to the solution of the HCl acid. This retro-BR does not exhibit a photo-activity. On the other hand an alteration of the π-electron conjugation in the excited state of the retinal might cause similar spectral features in the de-protonated state without covalent bond changes on the C₁₄. The solution mechanism of the charge separation along the retinal upon photoexcitation and the accompanying alteration of the π-electron conjugation was proposed (Chernavskii, D. S. An alternative model of the bacteriorhodopsin action and unusual properties of the K-610-intermediate. Biofizika (1994)) and further theoretically corroborated (Buda, F., de Groot, H. J. M. & Bifone, A. Charge Localization and Dynamics in Rhodopsin. Phys. Rev. Lett. 77, 4474-4477 (1996)). Perhaps, we are observing the first experimental evidence of the proposed mechanism. It is interesting that contrary to other retinal proton pumps (BR, pSRII, PR) we don't see any additional intermediates (N or O-like) on the path of re-protonation. The MII (P₅) state directly converts to the ground state of NsXeR with a half-time of 27 (50 in lipid vesicles) milliseconds.

### Example 2—Crystallization of NsXeR

The NsXeR protein was prepared and purified as described in Example 1. Finally proteins were concentrated to 70 mg/ml for crystallization. NsXeR crystals grew in meso approach (Landau, E. M. & Rosenbusch, J. P. Lipidic cubic phases: A novel concept for the crystallization of membrane proteins. Proc. Natl. Acad. Sci. 93, 14532-14535 (1996); and Caffrey, M. & Cherezov, V. Crystallizing membrane proteins using lipidic mesophases. Nat. Protoc. 4, 706-731 (2009); each incorporated herein by reference), similar to that used in previous works (Gordeliy, V. I. et al. Molecular basis of transmembrane signalling by sensory rhodopsin II-transducer complex. Nature 419, 484-487 (2002); incorporated by reference). The solubilized protein in the crystallization buffer was mixed with premelted at 47° C. monoundecenoin (Nu-Chek Prep) to form a lipidic mesophase. 100 nl aliquots of a protein-mesophase mixture were spotted on a 96-well LCP glass sandwich plate (Marienfeld) and overlaid with 600 nL of precipitant solution by means of the NT8 crystallization robot (Formulatrix). The best crystals were obtained with a protein concentration of 20 mg/ml and 2.0 M sodium malonate, pH 8.0 (Hampton Research). The crystals were grown at 22° C. and appeared in 1-4 weeks.

X-ray diffraction data (wavelengths 0.969 and 0.972 Å) were collected at ID23-1 beamline of the ESRF at 100 K, with a PILATUS 6M detector. Diffraction images were processed with XDS (Kabsch, W. XDS. Acta Crystallogr. D Biol. Crystallogr. 66, 125-132 (2010); incorporated by reference). The reflection intensities were scaled with SCALA from the CCP4 suite (Winn, M. D. et al. Overview of the CCP 4 suite and current developments. Acta Crystallogr. D Biol. Crystallogr. 67, 235-242 (2011); incorporated by reference). Crystallographic data collection and refinement statistics is shown in the following table.

| Data collection | | |
|---|---|---|
| Space group | P2₁2₁2₁ | |
| Cell dimensions | | |
| a, b, c (Å) | 64.50, 94.71, 198.36 | |
| α, β, γ (°) | 90, 90, 90 | |
| Resolution (Å) | 47-3.4 | (3.67-3.4) |
| R_{merge} (%) | 17.5 | (150.2) |
| I/σI | 6.0 | (1.0) |
| Completeness (%) | 99.8 | (99.8) |
| Redundancy | 4.5 | (4.6) |

| | |
|---|---|
| Refinement | |
| Resolution (Å) | 47.37-3.4 |
| No. reflections | 31932 |
| R_{work}/R_{free} (%) | 28.5/30.8 |
| No. atoms | |
| Protein and retinal | 4991 |
| B factors (Å²) | |
| Protein and retinal | 107.6 |
| r.m.s. deviations | |
| Bond lengths (Å) | 0.011 |
| Bond angles (°) | 1.486 |

The structure was refined to the resolution of 3.4 Å. Reference model (archaerhodopsin-2, PDB 2EI4) for molecular replacement was chosen with the MoRDa pipeline (Vagin, A. & Lebedev, A. MoRDa, an automatic molecular replacement pipeline. Acta Crystallogr. Sect. Found. Adv. 71, s19-s19 (2015); incorporated by reference). Initial phases were successfully obtained in P2₁2₁2₁ space group by an Automated Model Building and Rebuilding using Autobuild (Adams, P. D. et al. PHENIX: a comprehensive Python-based system for macromolecular structure solution. Acta Crystallogr. D Biol. Crystallogr. 66, 213-221 (2010); incorporated by reference). The initial model was iteratively refined using REFMAC5 (Murshudov, G. N. et al. REFMAC 5 for the refinement of macromolecular crystal structures. Acta Crystallogr. D Biol. Crystallogr. 67, 355-367 (2011); incorporated by reference), PHENIX and Coot (Emsley, P. & Cowtan, K. Coot: model-building tools for molecular graphics. Acta Crystallogr. D Biol. Crystallogr. 60, 2126-2132 (2004); incorporated by reference).

P2₁2₁2₁ space group crystals contain one trimer of NsXeR in the asymmetric unit. Positions of the residues 95-97 in loop CD, 154-156 in loop EF are not resolved.

The light-driven inward proton pump XeR has seven transmembrane α-helices (A-G) and a co-factor retinal covalently bound to 213 Lysine via the Schiff base. The helix A is preceded with a small N-terminal α-helix, which is capping the protein on the extracellular side.

Comparison with BR structure (PDB 1C3W) indicates considerable differences in helices localization and form, A and G helices are significantly distorted, presumably due to the presence of prolines in those helices. Xenorhodopsins have a conservative residue Pro-209 (position in NsXeR), which is located at the position of Asp212 in BR. Our experiments showed that its replacement with Asp makes the protein unstable. If changed to glycine, the pumping activity dramatically decreases (see below table). Thus, Pro209 is crucial for proton pumping.

Proton translocation validation

| | | |
|---|---|---|
| H48X | | Not folded, cells not colored |
| D76E, D76N, | | Not folded, cells not colored |
| D76S, D76T | | |
| D220N | No pumping | Colored cells |
| D220E | Fully functional | |
| C212A, C212D | | Not folded, cells not colored |
| C212S | Lowered pumping | |

Conservative aminoacids mutations

| | | |
|---|---|---|
| W73R | | Not folded, cells not colored |
| W73A | Strongly lowered | Colored cells |
| | pumping | |
| W73Y | Lowered pumping | |
| S55A | | Not folded, cells not colored |
| P209G | Strongly lowered | |
| | pumping | |
| P209D | | Not folded, cells not colored |

N-terminus

| | | |
|---|---|---|
| Δ2-12 | Lowered pumping | Colored, not stable |
| | | when solubilized |
| E4Q | Fully functional | |
| E4A, E4L | Lowered pumping | |

Proton-Uptake Region and Active Center

Retinal is in 13-cis conformation. However, due to insufficient resolution we cannot distinguish whether it is in 15-syn or 15-anti conformation. NsXeR has a big proton-uptake cavity, which is separated from the bulk with an N-term very short helix on the extracellular part of the protein. We suggest that the cavity is filled with water molecules. The putative proton donor Asp76 might be available from that cavity. Mutations of Asp76 to Glu, Ser, Thr and Asn do not allow the protein to fold correctly (mutants were not colored, see above table). This is evidence of not only functional, but also significant structural role of these amino acids. Ser55 is located close to Asp76 and it may stabilize this residue. Substitution of Ser55 with Alanine (Ala53 in BR) also breaks protein folding.

Residues Tyr3 from N-terminal helix and Trp73, which is the analogue of highly conservative amino acid Arg82 (position in BR), separate the proton-uptake cavity from the bulk of the extracellular part of the protein, so that the proton may enter the protein through the space between the helices A and B and loop BC. Substitution of Trp73 with Arg was fatal for protein folding (W73R mutant is not colored). W73A mutant binds retinal and has the color of the wild-type protein, but demonstrates no pumping activity, which means this residue is critical for proton translocation.

Proton-Release Region

Another major difference of NsXeR from other known microbial retinal proton pumps is that it has no charged amino acid at the position equivalent to Asp96 in BR (in NsXeR it is Ala71). However, the residues His48 (10 Å from the Schiff base in the ground state) and Asp220 (12 Å), which are connected via a hydrogen bond are located close to the expected proton acceptor position. Substitution of Asp220 with Asn demolishes proton pumping completely.

His48 is a unique residue, which is not present at a similar place in other known microbial rhodopsins. Our experiments showed that substitution of Histidine-48 with any other amino acid crushes protein structure (all mutants are not colored), which indicated its crucial role in protein architecture. We suggest that the pair His48-Asp220 is a proton acceptor, and the protonation processes from the Schiff base through the His48 residue, more precisely, through the pair His48-Asp220.

Remarkably, it is exactly the same proton acceptor pair as in proteorhodopsins. However, contrary to XeRs it is placed at the extracellular part of the protein close to the Schiff base and serves as a Schiff base proton acceptor, which is accessible from the bulk through a big proton-release cavity, so a further proton release may easily proceed directly to the bulk along the proton gradient. Thus, a unique and unusual set of key residues in NsXeR results in inwardly directed proton pumping

Putative Mechanism of Inwardly Directed Proton Transport

The structure and experiments with mutated amino acids provide insights into the mechanism of inwardly directed proton transport. Upon illumination retinal isomerizes and the Schiff base which is surrounded by a hydrophobic environment deprotonates and the proton is translocated to the deprotonated His48-Asp220 pair. It happens in MI and MII intermediate states since both intermediates correspond to the deprotonated state of the retinal Schiff base. Indeed, it is known that Asp-His interaction substantially lowers the pKₐ of Asp by stabilizing its deprotonated state. A key role of the Asp-His pair in proton translocation is supported by the mentioned above experiments with the mutated Asp and His. We suggest that after re-isomerization of the retinal the protonated Asp-His pair, connected to a hydrophilic cavity (“proton release cavity”, releases a proton directly to the cytoplasm. After isomerization of the retinal Asp76 protonates through the hydrophilic cavity. Re-isomerization of the retinal results also in re-protonation of the Schiff base from D76.

### Example 3—Optogenetic Implications

Experiments with Human Embryonic Kidney (HEK) and Neuroblastoma Glioma (NG) Cells

The human codon optimized NsXeR gene was synthesized commercially (Eurofins). The gene was cloned into the pcDNA3.1(−) vector bearing an additional membrane trafficking signal (Gradinaru, V. et al. Molecular and Cellular Approaches for Diversifying and Extending Optogenetics. Cell 141, 154-165 (2010), incorporated herein by reference), a P2A self-cleaving peptide (Kuzmich, A. I., Vvedenskii, A. V., Kopantzev, E. P. & Vinogradova, T. V. Quantitative comparison of gene co-expression in a bicistronic vector harboring IRES or coding sequence of porcine teschovirus 2A peptide. Russ. J. Bioorganic Chem. 39, 406-416 (2013); incorporated by reference) and a GFP variant at the C-terminus (Shcherbo, D. et al. Bright far-red fluorescent protein for whole-body imaging. Nat. Methods 4, 741-746 (2007); incorporated by reference). The gene was cloned under the CMV promoter. The sequence was verified by sequencing.

The HEK293 and NG108-15 cells at confluency of 80% were transfected with the plasmid and Lipofectamine LTX according to the manufacturer's protocol (ThermoFisher Scientific, USA). The cells were incubated under 5% CO₂ at 37° C. for two days before measurements.

For the electrophysiological characterization of NsXeR whole cell patch-clamp recordings were performed (Axopatch 200B interface, Axon Instruments). Patch pipettes with resistances of 2-5 MΩ were fabricated from thin-walled borosilicate glass (GB150F-8P) on a horizontal puller (Model P-1000, Sutter Instruments). For experiments in HEK293 cells the pipette solution contained 110 mM NaCl, 2 mM MgCl₂, 10 mM EGTA, 10 mM HEPES, pH 7.4 (pipette solution 1) and the bath solution contained 140 mM NaCl, 2 mM MgCl₂, 10 mM HEPES, pH 7.4 (bath solution 1). For the experiments in NG108-15 cells the pipette solution contained 110 mM Na₂SO₄, 4 mM MgSO₄, 10 mM EGTA, 10 mM HEPES, pH 7.4 (with H₂SO₄) (pipette solution 2) and the bath solution contained 140 mM N-methyl-D-glucamine, 4 mM MgSO₄, 10 mM HEPES, pH 7.4 (with H₂SO₄) (bath solution 2).

Photocurrents were measured in response to light pulses with a saturating intensity of 23 mW/mm² using diode-pumped solid-state lasers (λ=532 nm) focused into a 400-μm optic fiber. Light pulses were applied by a fast computer-controlled shutter (Uniblitz LS6ZM2, Vincent Associates). Ultrashort nanosecond light pulses were generated by the Opolette 355 tunable laser system (OPTOPRIM). For the measurement of the actionspectra the pulse energies at the different wavelengths were set to values which corresponded to equal photon counts of 10¹⁹ photons/m². Moreover photocurrent-voltage relationships at membrane potentials ranging from −100 mV to +60 mV were measured (except for On/Off kinetics, where membrane potentials ranged from −80 mV to +80 mV).

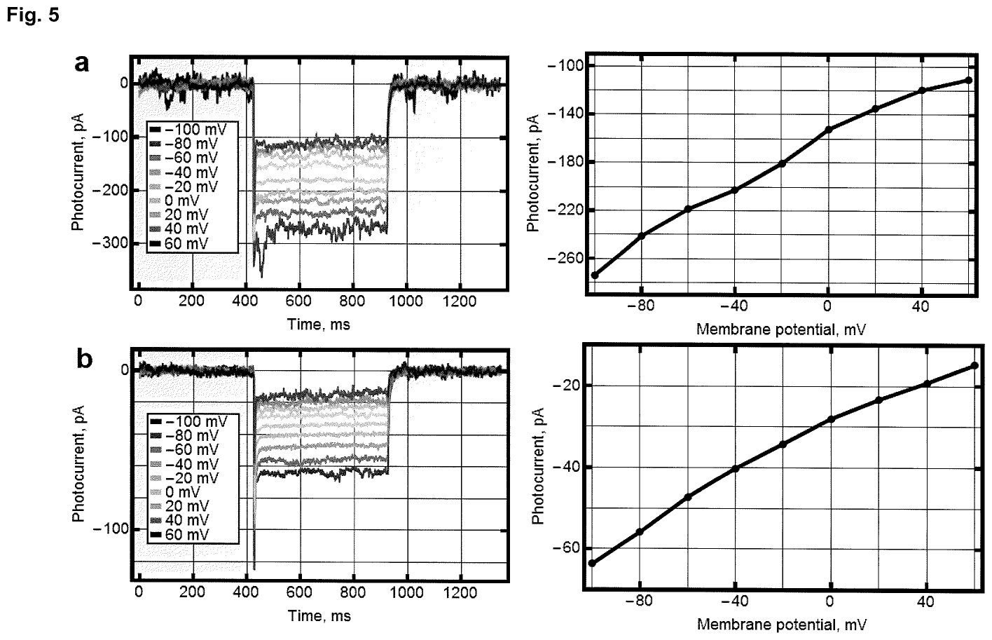

FIG. 5a shows photocurrents generated by NsXeR in the HEK293 cell. Typical photocurrent values vary from 40 to 150 pA at −60 mV applied potential, whereas the currents normalized to the capacitance (meaning the size) of the cell are about 1-2 pA/pF. An additional control experiment in NG108-15 cells was performed. To exclude the transport of Cl⁻ ions (which may account for apparent “inward” current) chloride salts in buffers were replaced by sulfate. To exclude monovalent ion transport into the cell we replaced Na⁺ in the bath solution by large N-methyl-D-glucamine. The pH of the solutions was symmetric (pH 7.4). However, similar photocurrents were recorded in this experimental configuration (FIG. 5b), convincing us that the transport of protons is responsible for the effect. Thus, the experiments with HEK and NG cells also confirm that NsXeR is an inwardly directed pump and show that it is able to generate significant currents through plasma membranes upon illumination of the cells.

Light-Triggered Spiking in Rat Hippocampal Neurons

We heterologously expressed NsXeR in rat hippocampal neurons by means of adeno-associated virus mediated gene transfer. Hippocampi were isolated from postnatal P1 Sprague-Dawley rats and treated with papain (20 U ml⁻¹) for 20 min at 37° C. The hippocampi were washed with DMEM (Invitrogen/Gibco, high glucose) supplemented with 10% fetal bovine serum and titrated in a small volume of this solution. ˜96,000 cells were plated on poly-D-lysine/laminin coated glass cover slips in 24-well plates. After 3 hours the plating medium was replaced by culture medium (Neurobasal A containing 2% B-27 supplement, and 2 mM Glutamax-I).

rAAV2/1 virus was prepared using a pAAV2 vector with a human synapsin promoter containing the humanized DNA sequence of NsXeR, C-terminally fused to the Kir2.1 membrane trafficking signal, a P2A self-cleaving peptide and a GFP variant. Briefly 5×10⁹ genome copies/ml (GC/ml) of rAAV2/1 virus was added to each well 4-9 days after plating. The electrophysiological recordings were performed 19-23 days after transduction.

For the electrophysiological characterization we performed a whole cell patch clamp experiments under the current clamp conditions. Briefly, patch pipettes with resistances of 3-8 MΩ were filled with 129 mM potassium gluconate, 10 mM HEPES, 10 mM KCl, 4 mM MgATP and 0.3 mM Na₃GTP, titrated to pH 7.3. The extracellular solution contained 125 mM NaCl, 2 mM KCl, 2 mM CaCl₂, 1 mM MgCl₂, 30 mM glucose and 25 mM HEPES, titrated to pH 7.3. Electrophysiological signals were filtered at 10 kHz, digitized with an Axon Digidata 1322A (50 kHz) and acquired and analyzed using pClamp9 software (Axon Instruments).

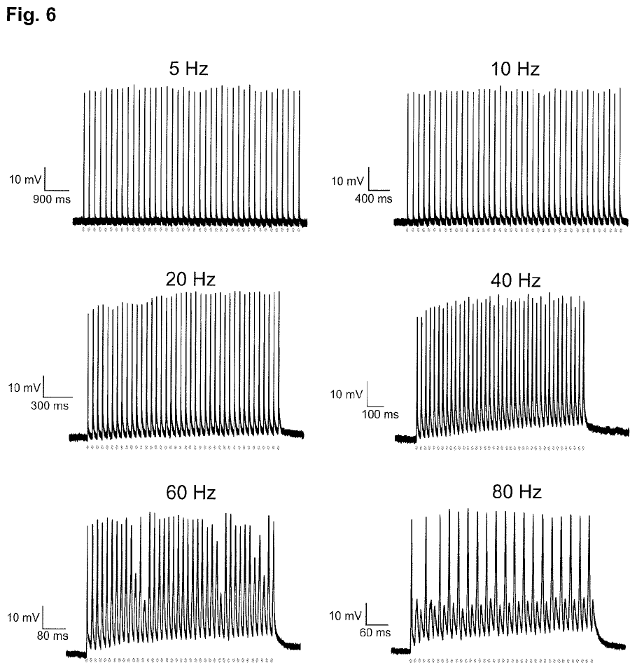

The NsXeR-mediated, light-triggered inward transport of protons led to the depolarization of the membrane potential. Therefore, light-triggered spiking in rat hippocampal neurons was possible (FIG. 6). NsXeR enabled a fast, neural photostimulation with a firing success rate of 100% up to a frequency of 40 Hz. Spike failures at higher stimulation frequencies can be explained by intrinsic properties of the rat hippocampal neurons as a vast majority of rat hippocampal neurons have a maximal firing frequency of 40-60 Hz (Gunaydin, L. A. et al. Ultrafast optogenetic control. Nat. Neurosci. 13, 387-392 (2010)).

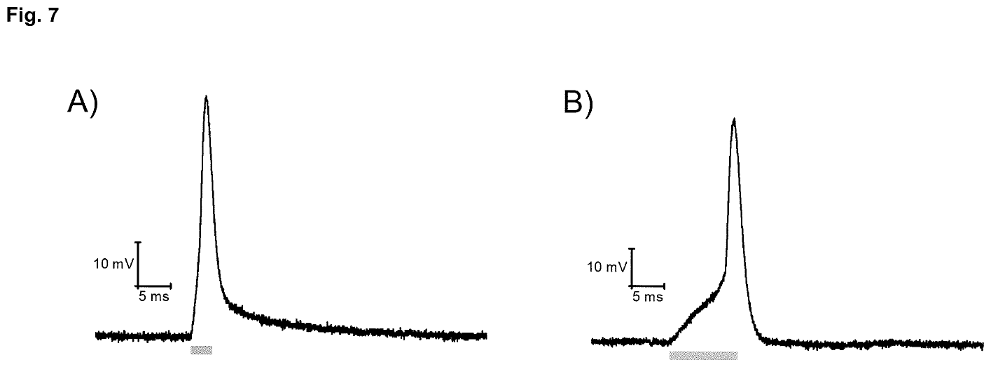

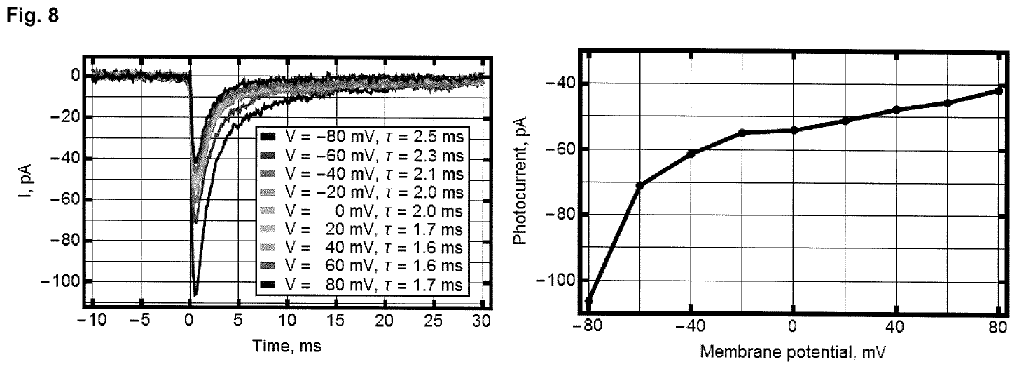

An important observation is that light-triggered spiking could be achieved with a pulse width of only 3 ms (FIG. 7a), which approximately corresponds to the turnover time of the pump (FIG. 8). Hence, the extent of depolarization due to the transport of a single proton by each NsXeR is sufficient to successfully trigger action potentials. However a variability of the spike latencies was observed (FIG. 7), which in some cases required longer pulse widths for the light-triggered spiking (FIG. 7b). Longer spike latencies could be explained by a comparatively lower expression of NsXeR in those neurons.

### Example 4—Optogenetic Stimulation of the Auditory Pathway

Hernandez et al. J Clin Invest. 124, 1114-1129 (2014) (incorporated herein by reference), demonstrates a strategy for optogenetic stimulation of the auditory pathway in rodents. In particular, the authors describe animal models to characterize optogenetic stimulation, which is the optical stimulation of neurons genetically engineered to express the light-gated ion channel channelrhodopsin-2 (ChR2). Optogenetic stimulation of spiral ganglion neurons (SGNs) activates the auditory pathway, as demonstrated by recordings of single neuron and neuronal population responses. Furthermore, optogenetic stimulation of SGNs restore auditory activity in deaf mice. Approximation of the spatial spread of cochlear excitation by recording local field potentials (LFPs) in the inferior colliculus in response to suprathreshold optical, acoustic, and electrical stimuli indicate that optogenetic stimulation achieves better frequency resolution than monopolar electrical stimulation.

Introducing the coding sequence for the light-inducible inward proton pump of the present disclosure, such as NsXeR, into the constructs as described, e.g., by Hernandez et al. represents routine practice.

### Example 5—Optogenetic Approach for the Recovery of Vision

Macé et al. Mol Ther. 23, 7-16 (2015) (incorporated herein by reference), describes optogenetic reactivation of retinal neurons mediated by adeno-associated virus (AAV) gene therapy. Most inherited retinal dystrophies display progressive photoreceptor cell degeneration leading to severe visual impairment. Optogenetic reactivation of retinal neurons mediated by adeno-associated virus (AAV) gene therapy has the potential to restore vision regardless of patient-specific mutations. The challenge for clinical translatability is to restore a vision as close to natural vision as possible, while using a surgically safe delivery route for the fragile degenerated retina. To preserve the visual processing of the inner retina, ON bipolar cells are targeted, which are still present at late stages of disease. For safe gene delivery, a recently engineered AAV variant is used that can transduce the bipolar cells after injection into the eye's easily accessible vitreous humor. It is shown that AAV encoding channelrhodopsin under the ON bipolar cell-specific promoter mediates long-term gene delivery restricted to ON-bipolar cells after intravitreal administration. Channelrhodopsin expression in ON bipolar cells leads to restoration of ON and OFF responses at the retinal and cortical levels. Moreover, light-induced locomotory behavior is restored in treated blind mice.

Introducing the coding sequence for the light-inducible inward proton pump of the present disclosure, such as NsXeR, into the constructs as described, e.g., by Mace et al. represents routine practice. The new light-inducible inward proton pumps of the present disclosure are inserted in the cassettes for the activation of ON bipolar cells as well as for the Ganglion cells in the retina.

### LIST OF REFERENCES

Oesterhelt, D. & Stoeckenius, W. Rhodopsin-like Protein from the Purple Membrane of Halobacterium halobium. Nature 233, 149-152 (1971).
Ernst, O. P. et al. Microbial and Animal Rhodopsins: Structures, Functions, and Molecular Mechanisms. Chem. Rev. 114, 126-163 (2014).
Ugalde, J. A., Podell, S., Narasingarao, P. & Allen, E. E. Xenorhodopsins, an enigmatic new class of microbial rhodopsins horizontally transferred between archaea and bacteria. Biol. Direct 6, 52 (2011).
Ghai, R. et al. New Abundant Microbial Groups in Aquatic Hypersaline Environments. Sci. Rep. 1, (2011).
Vavourakis, C. D. et al. Metagenomic Insights into the Uncultured Diversity and Physiology of Microbes in Four Hypersaline Soda Lake Brines. Front. Microbiol. 7, (2016).
Gushchin, I. et al. Crystal structure of a light-driven sodium pump. Nat. Struct. Mol. Biol. 22, 390-395 (2015).
Studier, F. W. Protein production by auto-induction in high-density shaking cultures. Protein Expr. Purif. 41, 207-234 (2005).
Huang, K. S., Bayley, H. & Khorana, H. G. Delipidation of bacteriorhodopsin and reconstitution with exogenous phospholipid. Proc. Natl. Acad. Sci. 77, 323-327 (1980).
Racker, E. & Stoeckenius, W. Reconstitution of purple membrane vesicles catalyzing light-driven proton uptake and adenosine triphosphate formation. J. Biol. Chem. 249, 662-663 (1974).
Ritchie, T. K. et al. in Methods in Enzymology (ed. Düzgünes, N.) 464, 211-231 (Academic Press, 2009).
Chizhov, I. et al. Spectrally silent transitions in the bacteriorhodopsin photocycle. Biophys. J. 71, 2329-2345 (1996).
Gordeliy, V. I. et al. Molecular basis of transmembrane signalling by sensory rhodopsin II-transducer complex. Nature 419, 484-487 (2002).
Chernavskii, D. S. An alternative model of the bacteriorhodopsin action and unusual properties of the K-610-intermediate. Biofizika (1994).
Buda, F., de Groot, H. J. M. & Bifone, A. Charge Localization and Dynamics in Rhodopsin. Phys. Rev. Lett. 77, 4474-4477 (1996).
Landau, E. M. & Rosenbusch, J. P. Lipidic cubic phases: A novel concept for the crystallization of membrane proteins. Proc. Natl. Acad. Sci. 93, 14532-14535 (1996).
Caffrey, M. & Cherezov, V. Crystallizing membrane proteins using lipidic mesophases. Nat. Protoc. 4, 706-731 (2009).
Kabsch, W. XDS. Acta Crystallogr. D Biol. Crystallogr. 66, 125-132 (2010).
Winn, M. D. et al. Overview of the CCP 4 suite and current developments. Acta Crystallogr. D Biol. Crystallogr. 67, 235-242 (2011).
Vagin, A. & Lebedev, A. MoRDa, an automatic molecular replacement pipeline. Acta Crystallogr. Sect. Found. Adv. 71, s19-s19 (2015).
Adams, P. D. et al. PHENIX: a comprehensive Python-based system for macromolecular structure solution. Acta Crystallogr. D Biol. Crystallogr. 66, 213-221 (2010).
Murshudov, G. N. et al. REFMAC 5 for the refinement of macromolecular crystal structures. Acta Crystallogr. D Biol. Crystallogr. 67, 355-367 (2011).
Emsley, P. & Cowtan, K. Coot: model-building tools for molecular graphics. Acta Crystallogr. D Biol. Crystallogr. 60, 2126-2132 (2004).
Gradinaru, V. et al. Molecular and Cellular Approaches for Diversifying and Extending Optogenetics. Cell 141, 154-165 (2010).
Kuzmich, A. I., Vvedenskii, A. V., Kopantzev, E. P. & Vinogradova, T. V. Quantitative comparison of gene co-expression in a bicistronic vector harboring IRES or coding sequence of porcine teschovirus 2A peptide. Russ. J. Bioorganic Chem. 39, 406-416 (2013).
Shcherbo, D. et al. Bright far-red fluorescent protein for whole-body imaging. Nat. Methods 4, 741-746 (2007).
Gunaydin, L. A. et al. Ultrafast optogenetic control. Nat. Neurosci. 13, 387-392 (2010).
Hernandez et al. Optogenetic stimulation of the auditory pathway. J Clin Invest. 124, 1114-1129 (2014).
Macé et al. Targeting channelrhodopsin-2 to ON-bipolar cells with vitreally administered AAV Restores ON and OFF visual responses in blind mice. Mol Ther. 23, 7-16 (2015).
Kawanabe et al. Engineering an inward proton transport from a bacterial sensor rhodopsin. J Am Chem Soc. 131, 16439-16444 (2009).
Kawanabe et al. An inward proton transport using Anabaena sensory rhodopsin. J Microbiol. 49, 1-6 (2011).
Dong et al. Structure of an Inward Proton-Transporting Anabaena Sensory Rhodopsin Mutant: Mechanistic Insights. Biophys J. 111(5), 963-972 (September 2016).
Inoue et al. Converting a light-driven proton pump into a light-gated proton channel. J Am Chem Soc. 137, 3291-3299 (2015).
Inoue et al. A natural light-driven inward proton pump. Nat Commun. 7, 13415 (November 2016).

1-15. (canceled)

16. A method of medical treatment, comprising administering a light-driven inward directed proton pump having at least 59% sequence similarity over the full length of SEQ ID NO: 1 (NsXeR) to a patient.

17. The method of claim 16, wherein the light-driven inward directed proton pump has at least 90% sequence similarity to the full length of SEQ ID NO: 1 (NsXeR).

18. The method of claim 16, wherein the light-driven inward directed proton pump has at least 90% sequence identity to the full length of SEQ ID NO: 1 (NsXeR).

19. The method of claim 16, wherein light-driven inward directed proton pump is not mutated at position E4, H48, S55, W73, D76, S80, A87, P209, C212, K214, and D220 of SEQ ID NO: 1; or wherein light-driven inward directed proton pump is not truncated at the N-terminus; or wherein light-driven inward directed proton pump is not mutated at position E4, H48, S55, W73, D76, S80, A87, P209, C212, K214, and D220 of SEQ ID NO: 1, and is not truncated at the N-terminus.

20. The method of claim 16, wherein the light-driven inward directed proton pump comprises an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5).

21. The method of claim 16, wherein the light-driven inward directed proton pump consists of an amino acid sequence selected from SEQ ID NO: 1 (NsXeR), 2 (HrvXeR1), 9 (HrvXeR), 10 (AlkXeR), 11 (AlkXeR1), 12 (AlkXeR2), 13 (AlkXeR3), 14 (AlkXeR4), and 15 (AlkXeR5).

22. The method of claim 16, wherein the light-driven inward directed proton pump is active between pH 6 and pH 8.

23. The method of claim 16, wherein the absorption maximum of the light-driven inward directed proton pump is between 560 nm and 580 nm.

24. The method of claim 16, wherein the photocycle of the light-driven inward directed proton pump is less than 50 ms, if measured in proteo-nanodiscs exhibiting a molar ratio of DMPC:MSP1E3:light-driven inward directed proton pump of 100:2:3 at 20° C. and pH 7.5, providing pulses of 5 ns duration at 532 nm wavelength and energy of 3 mJ/pulse.

25. The method of claim 16, wherein the light-driven inward directed proton pump has a turnover rate of more than 250 s⁻¹, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl2, 1 mM MgCl₂, 30 mM glucose and 25 mM HEPES, titrated to pH 7.3.

26. The method of claim 16, wherein the light-driven inward directed proton pump is capable of triggering action potentials in a frequency of more than 40 Hz, if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.

27. The method of claim 16, wherein the light-driven inward directed proton pump is capable of being triggered with a pulse width of 3 ms of λ=532 nm and an intensity of 23 mW/mm², if measured in rat hippocampal neurons by patch-clamp measurements in the whole cell configuration using patch pipettes with resistances of 3-8 MΩ, filled with 129 mM potassium gluconate, 10 mM HEPES, 10 mM KCl, 4 mM MgATP and 0.3 mM Na₃GTP, titrated to pH 7.3, and an extracellular solution contained 125 mM NaCl, 2 mM KCl, 2 mM CaCl₂, 1 mM MgCl₂, 1 mM MgCl₂, 30 mM glucose and 25 mM HEPES, titrated to pH 7.3.

28. The method of claim 16, wherein the medical treatment is selected from restoring auditory activity, recovery of vision, treating or alleviating alkalosis, treating or alleviating neurological injury, treating or alleviating brain damage, treating or alleviating seizure, and treating or alleviating a degenerative neurological disorder.

29. The method of claim 28, wherein the neurological disorder is Parkinson's disease or Alzheimer's disease.

30. A nucleic acid construct, comprising a nucleotide sequence coding for a light-driven inward directed proton pump having at least 59% sequence similarity over the full length of SEQ ID NO: 1 (NsXeR), wherein the nucleotide sequence is codon-optimized for expression in human cells.

31. An expression vector, comprising a nucleotide sequence coding for a light-driven inward directed proton pump having at least 59% sequence similarity over the full length of SEQ ID NO: 1 (NsXeR), wherein the nucleotide sequence is optimized for expression in human cells.

32. The expression vector of claim 31, wherein the vector is a viral vector.

33. The expression vector of claim 31, wherein the coding sequence of the light-driven inward directed proton pump is under the control of a neuronal cell specific human promotor.

34. The expression vector of claim 33, wherein the neuronal cell specific human promotor is the human synapsin promotor.

35. A mammalian cell expressing a light-driven inward directed proton pump having at least 59% sequence similarity over the full length of SEQ ID NO: 1 (NsXeR).

36. A mammalian cell comprising a nucleic acid construct according to claim 30 an expression vector according to claim 31.

37. The mammalian cell of claim 36, wherein the cell is
(a) a hippocampal cell, a photoreceptor cell, a retinal rod cell, a retinal cone cell, a retinal ganglion cell, a bipolar neuron, a ganglion cell, a pseudounipolar neuron, a multipolar neuron, a pyramidal neuron, a Purkinje cell, or a granule cell; or
(b) a neuroblastoma cell; a HEK293 cell; a COS cell; a BHK cell; a CHO cell; a myeloma cell; or a MDCK cell.

38. A liposome, comprising a light-driven inward directed proton pump having at least 59% sequence similarity over the full length of SEQ ID NO: 1 (NsXeR).

39. A non-human mammal, comprising a cell according to claim 35.

40. The non-human animal of claim 39, wherein the cell is an endogenous cell.


SEQUENCE LISTING (16 sequences)

SEQ ID NO: 1 - protein, 228 aa, Nanosalina sp. (strain J07AB43)
MVYEAITAGGFGSQPFILAYIITAMISGLLFLYLPRKLDVPQKFGIIHFFIVVWSGLMYTNFLNQSFLSDYAWYMDWMVSTPLILLALGLTAFHGADTKRYDLLGALLGAEFTLVITGLLAQAQGSITPYYVGVLLLLGVVYLLAKPFREIAEESSDGLARAYKILAGYIGIFFLSYPTVWYISGIDALPGSLNILDPTQTSIALVVLPFFCKQVYGFLDMYLIHKAE

SEQ ID NO: 2 - protein, 229 aa, Haloredivivus sp. (strain G17)
MVYEAIAASGSTPYLMAYIATAFLSGLLYLFLRKVWWTNVPLKFPIIHFFIVTWSGIMYLNFLNGTALSDFGWYMDWMISTPLILLALGLTAMHGRETRWDLLGALMGLQFMLVITGIISQESGMTYAYWIGNALLLGVFYLVWGPLREMAKETSDVLARSYTTLSAYISVFFVLYPTVWYLSETIYPAGPGIFGAFETSVAFVILPFFCKQAYGFLDMYLIHEAEEQM

SEQ ID NO: 3 - protein, 261 aa, Nostoc sp. (strain PCC 7120 / SAG 25.82 / UTEX 2576)
MNLESLLHWIYVAGMTIGALHFWSLSRNPRGVPQYEYLVAMFIPIWSGLAYMAMAIDQGKVEAAGQIAHYARYIDWMVTTPLLLLSLSWTAMQFIKKDWTLIGFLMSTQIVVITSGLIADLSERDWVRYLWYICGVCAFLIILWGIWNPLRAKTRTQSSELANLYDKLVTYFTVLWIGYPIVWIIGPSGFGWINQTIDTFLFCLLPFFSKVGFSFLDLHGLRNLNDSRQTTGDRFAENTLQFVENITLFANSRRQQSRRRV

SEQ ID NO: 4 - protein, 262 aa, Halobacterium salinarum (strain ATCC 700922 / JCM 11081 / NRC-1) (Halobacterium halobium)
MLELLPTAVEGVSQAQITGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGEAEAPEPSAGDGAAATSD

SEQ ID NO: 5 - protein, 249 aa, Gamma-proteobacterium EBAC31A08
MKLLLILGSVIALPTFAAGGGDLDASDYTGVSFWLVTAALLASTVFFFVERDRVSAKWKTSLTVSGLVTGIAFWHYMYMRGVWIETGDSPTVFRYIDWLLTVPLLICEFYLILAAATNVAGSLFKKLLVGSLVMLVFGYMGEAGIMAAWPAFIIGCLAWVYMIYELWAGEGKSACNTASPAVQSAYNTMMYIIIFGWAIYPVGYFTGYLMGDGGSALNLNLIYNLADFVNKILFGLIIWNVAVKESSNA

SEQ ID NO: 6 - protein, 291 aa, Natronomonas pharaonis (Natronobacterium pharaonis)
MTETLPPVTESAVALQAEVTQRELFEFVLNDPLLASSLYINIALAGLSILLFVFMTRGLDDPRAKLIAVSTILVPVVSIASYTGLASGLTISVLEMPAGHFAEGSSVMLGGEEVDGVVTMWGRYLTWALSTPMILLALGLLAGSNATKLFTAITFDIAMCVTGLAAALTTSSHLMRWFWYAISCACFLVVLYILLVEWAQDAKAAGTADMFNTLKLLTVVMWLGYPIVWALGVEGIAVLPVGVTSWGYSFLDIVAKYIFAFLLLNYLTSNESVVSGSILDVPSASGTPADD

SEQ ID NO: 7 - protein, 280 aa, Dokdonia eikasta
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNKELVEANS

SEQ ID NO: 8 - protein, 239 aa, Natronomonas pharaonis (Natronobacterium pharaonis)
MVGLTTLFWLGAIGMLVGTLAFAWAGRDAGSGERRYYVTLVGISGIAAVAYVVMALGVGWVPVAERTVFAPRYIDWILTTPLIVYFLGLLAGLDSREFGIVITLNTVVMLAGFAGAMVPGIERYALFGMGAVAFLGLVYYLVGPMTESASQRSSGIKSLYVRLRNLTVILWAIYPFIWLLGPPGVALLTPTVDVALIVYLDLVTKVGFGFIALDAAATLRAEHGESLAGVDTDAPAVAD

SEQ ID NO: 9 - protein, 225 aa, Haloredivivus sp.
MVFEAIAGSGTEMYIQAYIATAFLSGLLYLYLSRVWWDNVPLKFPIVHFFIVTWSGIMYLNFLNESLFSNFAWYMDWLISTPLIVLALGMTALHHADKKHYDLLGMLMGLQFMLVVTGIISQSTGATLAYWVGNALLLGVIYLLWFPFREIAEQGSERLAKSYKTLAAYISIFFVLYPAAWYLGTPGPMEVLSDFQTSLAFVVLPFFCKQVYGFLDLYMIHHAED

SEQ ID NO: 10 - protein, 230 aa, Alkalimonas
MVLPELATLTSQTIAAYIAATALSAVAFLWMSKNWGDVPKKFYLIHFFIVSWSGLMYMNILYDTSIAELAFYADWLVSTPLIVLALGLSAYIASDSTDWSMVGSLMGLQFMLIAAGLLAHVAETAAATWAFYGISCLFMFGVIYMIWGPLMRVTESNDALNREYHKLGLFVILTWLSYPTIWALGDVGGYGLGVLSDYQVTLGYVILPFLCKAGFGFLDIYLLDRISDDI

SEQ ID NO: 11 - protein, 232 aa, Alkalimonas
MVYEAIAGSGSSPYIWAYIVTAFLSGLAFLYLSRVWDNVPRRFPIVHFFIVTWSGLMYLNFVEGQTILSNYAWYVDWMVSTPLIVLALALTATYKSEKNHYDLIAALMGLQFMLIVTGIISQEAAASTAYAFWIGCGLLAGVAYLLWVPFRKIAEETSEVLAKKYKLLAGYITVFFALYPLVWYLSGTVYPSGPGMLGAFETSLAFVILPFFCKQVYGFLDMYLIHKAGEDL

SEQ ID NO: 12 - protein, 232 aa, Alkalimonas
MVYEAIAASGSSPYIWAYIITAFLSGLAFLYLSRIWDNVPRRFPIVHFFIVTWSGLMYLNFVEGQTLISDYAWYVDWMISTPLIVLALAMTATYKSEKNHYDLIAALMGLQFMLIVTGIISQEAAASTAYAFWIGCGLLAGVAYLLWVPFRKIAEETSDVLAKKYKLLAGYITVFFALYPAAWYLSEVVYPEGPAMLGAFETSLAFVILPFFCKQVYGFLDMYLIQKAGEEI

SEQ ID NO: 13 - protein, 252 aa, Alkalimonas
MIGVILIYEVTSRLFMVYEAIAASGSSPYIWAYIATALLSGLAYLFLYRVWDNVPRRFPIIHFFIVSWSALMYLSFVEGQTLFSDYVWYMDWIISTPLIVLALVLTATYKSEGSHYDLIGAAMGLQFMLIVTGIVSQDTAMSADFVGIPVAFWLGCVWLAGLIYLLWGPFKEIAEQTSHHLAQKYKILAGYISLFFALYPTAWYLSETVYPEGPAVLGAFETSLAFVILPFFCKQVYGFLDMYMIHQAGEEM

SEQ ID NO: 14 - protein, 232 aa, Alkalimonas; identical to SEQ ID NO: 12

SEQ ID NO: 15 - protein, 232 aa, Alkalimonas
MVYEAIAASGSSPYIWAYIATAFLSGLAFLYLSKVWDNVPRRFPIVHFFIVTWSGLMYLNFVEGQTLISDYAWYVDWMVSTPLIVLALALTATYKSEKNHYDLIGALMGLQFMLVVTGIISQEAAATTAYAFWIGCGLLVGVAYLLWVPFRKIAEETSEVLAKKYKILAGYITVFFALYPLVWYLSGTVYPEGPGMLGAFETSLAFVILPFFCKQVYGFLDMYLIQKAGKEL

SEQ ID NO: 16 - DNA, 684 bp, Artificial
  human codon-optimized NsXeR
atggtgtacgaggccatcacagccggcggattcggcagccagcctttcatcctggcctacatcatcaccgccatgatcagcggcctgctgttcctgtacctgccccggaagctggacgtgccccagaagttcggcatcatccactttttcatcgtcgtgtggagcggcctgatgtataccaacttcctgaaccagagcttcctgagcgactacgcctggtacatggactggatggtgtccacccccctgatcctgctggccctgggactgacagctttccacggcgccgacaccaagagatacgacctgctgggagcactgctgggcgccgagtttaccctcgtgatcactggactgctggctcaggcccagggctccatcaccccttactatgtgggcgtgctcctgctgctgggggtggtgtatctgctggccaagcccttcagagagatcgccgaggaaagcagcgacggcctggccagagcctacaagatcctggccggctatatcggcatcttctttctgtcctaccccaccgtgtggtacatcagcggcatcgacgccctgcccggcagcctgaatatcctggaccctacccagacctctatcgccctggtggtgctgccattcttctgtaaacaagtgtacggcttcctggacatgtacctgatccacaaggctgag